SEQUENCE LISTING (75 sequences)

SEQ ID NO: 1 - protein, 118 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 2 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

SEQ ID NO: 3 - protein, 118 aa, synthetic construct
QVELVESGGGVVQPGRSQRLSCAASGFTFSSTGMHWVRQAPGKGLEWVAYIWFDGSSTYYADSVRGRFTISRDNSKNTLYLQMNSLRAEDTAVYFCARELNRRYFDLWGRGTLVSVSS

SEQ ID NO: 4 - protein, 108 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKYPPWTFGQGTKVESK

SEQ ID NO: 5 - protein, 118 aa, synthetic construct
QVELVESGGGVVQPGRSQRLSCAASGFTFSSHGMHWVRQAPGKGLEWVAIIAGDASTTYYADSVRGRFTISRDNSKNTLYLQMNSLRAEDTAVYFCARELGRRYFDLWGRGTLVSVSS

SEQ ID NO: 6 - protein, 108 aa, synthetic construct; identical to SEQ ID NO: 4

SEQ ID NO: 7 - protein, 118 aa, synthetic construct; identical to SEQ ID NO: 1

SEQ ID NO: 8 - protein, 108 aa, synthetic construct; identical to SEQ ID NO: 4

SEQ ID NO: 9 - protein, 118 aa, synthetic construct
QVELVESGGGVVQPGRSQRLSCAASGFTFSSYGMHWVRQAPGKGLEWVATIWFDGSSTYYADSVRGRFTISRDNSKNTLYLQMNSLRAEDTAVYFCARELGRRYFDLWGRGTLVSVSS

SEQ ID NO: 10 - protein, 108 aa, synthetic construct; identical to SEQ ID NO: 4

SEQ ID NO: 11 - protein, 118 aa, synthetic construct
QVELVESGGGVVQPGRSQRLSCAASGFTFSSYGMHWVRQAPGKGLEWVAIINFDGSSTYYADSVRGRFTISRDNSKNTLYLQMNSLRAEDTAVYFCARELGRRYFDLWGRGTLVSVSS

SEQ ID NO: 12 - protein, 108 aa, synthetic construct; identical to SEQ ID NO: 4

SEQ ID NO: 13 - protein, 118 aa, synthetic construct
QVELVESGGGVVQPGRSQRLSCAASGFTFSSAGMHWVRQAPGKGLEWVAIIWADGSSTYYADSVRGRFTISRDNSKNTLYLQMNSLRAEDTAVYFCARELGRRYFDLWGRGTLVSVSS

SEQ ID NO: 14 - protein, 108 aa, synthetic construct; identical to SEQ ID NO: 4

SEQ ID NO: 15 - protein, 118 aa, synthetic construct
QVELVESGGGVVQPGRSQRLSCAASGFTFSSYGMHWVRQAPGKGLEWVAYIWFDGSSTYYADSVRGRFTISRDNSKNTLYLQMNSLRAEDTAVYFCARELGRRYFDLWGRGTLVSVSS

SEQ ID NO: 16 - protein, 108 aa, synthetic construct; identical to SEQ ID NO: 4

SEQ ID NO: 17 - protein, 118 aa, synthetic construct
QVELVESGGGVVQPGRSQRLSCAASGFTFSSHGMHWVRQAPGKGLEWVAYIWFDGSSTYYADSVRGRFTISRDNSKNTLYLQMNSLRAEDTAVYFCARELGRRYFDLWGRGTLVSVSS

SEQ ID NO: 18 - protein, 108 aa, synthetic construct
EIVLTQSPATLSLSPGERATLSCRASQSVSSALAWYQQKPGQAPRLLIYDASKRATGIPARFSGSGSGTDFTLTISSLEPEDFAVYYCQQRSKYPPWTFGQGTKVESK

SEQ ID NO: 19 - protein, 118 aa, synthetic construct
QVELVESGGGVVQPGRSQRLSCAASGFTFSSHGMHWVRQAPGKGLEWVATIWFDGSSTYYADSVRGRFTISRDNSKNTLYLQMNSLRAEDTAVYFCARELGRRYFDLWGRGTLVSVSS

SEQ ID NO: 20 - protein, 108 aa, synthetic construct
EIVLTQSPATLSLSPGERATLSCRASQSVSSTLAWYQQKPGQAPRLLIYDASKRATGIPARFSGSGSGTDFTLTISSLEPEDFAVYYCQQRSKYPPWTFGQGTKVESK

SEQ ID NO: 21 - protein, 118 aa, synthetic construct
QVELVESGGGVVQPGRSQRLSCAASGFTFSSYGMHWVRQAPGKGLEWVATIWHDGSSTYYADSVRGRFTISRDNSKNTLYLQMNSLRAEDTAVYFCARELGRRYFDLWGRGTLVSVSS

SEQ ID NO: 22 - protein, 108 aa, synthetic construct
EIVLTQSPATLSLSPGERATLSCRASQSVSSALAWYQQKPGQAPRLLIYDASKRATGIPARFSGSGSGTDFTLTISSLEPEDFAVYYCQQRGKYPPWTFGQGTKVESK

SEQ ID NO: 23-29 - skipped (7 entries)

SEQ ID NO: 30 - protein, 5 aa, synthetic construct
SHGMH

SEQ ID NO: 31 - protein, 5 aa, synthetic construct
STGMH

SEQ ID NO: 32 - protein, 5 aa, synthetic construct
SAGMH

SEQ ID NO: 33 - protein, 5 aa, synthetic construct
SYGMH

SEQ ID NO: 34 - protein, 17 aa, synthetic construct
IIAGDASTTYYADSVRG

SEQ ID NO: 35 - protein, 17 aa, synthetic construct
YIWFDGSSTYYADSVRG

SEQ ID NO: 36 - protein, 17 aa, synthetic construct
IIWADGSSTYYADSVRG

SEQ ID NO: 37 - protein, 17 aa, synthetic construct
IINFDGSSTYYADSVRG

SEQ ID NO: 38 - protein, 17 aa, synthetic construct
IIWFDGSSTYYADSVRG

SEQ ID NO: 39 - protein, 17 aa, synthetic construct
TIWHDGSSTYYADSVRG

SEQ ID NO: 40 - protein, 17 aa, synthetic construct
TIWFDGSSTYYADSVRG

SEQ ID NO: 41 - protein, 9 aa, synthetic construct
ELGRRYFDL

SEQ ID NO: 42 - protein, 9 aa, synthetic construct
ELNRRYFDL

SEQ ID NO: 43 - protein, 11 aa, synthetic construct
RASQSVSSALA

SEQ ID NO: 44 - protein, 11 aa, synthetic construct
RASQSVSSTLA

SEQ ID NO: 45 - protein, 11 aa, synthetic construct
RASQSVSSYLA

SEQ ID NO: 46 - protein, 7 aa, synthetic construct
DASKRAT

SEQ ID NO: 47 - protein, 10 aa, synthetic construct
QQRGKYPPWT

SEQ ID NO: 48 - protein, 10 aa, synthetic construct
QQRSKYPPWT

SEQ ID NO: 49 - protein, 10 aa, synthetic construct
QQRSKWPPWT

SEQ ID NO: 50 - skipped

SEQ ID NO: 51 - protein, 448 aa, synthetic construct
QVELVESGGGVVQPGRSQRLSCAASGFTFSSHGMHWVRQAPGKGLEWVAYIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 215 aa, synthetic construct
EIVLTQSPATLSLSPGERATLSCRASQSVSSALAWYQQKPGQAPRLLIYDASKRATGIPARFSGSGSGTDFTLTISSLEPEDFAVYYCQQRSKYPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 448 aa, synthetic construct
QVELVESGGGVVQPGRSQRLSCAASGFTFSSTGMHWVRQAPGKGLEWVAYIWFDGSSTYYADSVRGRFTISRDNSKNTLYLQMNSLRAEDTAVYFCARELNRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 215 aa, synthetic construct
EIVLTQSPATLSLSPGERATLSCRASQSVSSYLAWYQQKPGQAPRLLIYDASKRATGIPARFSGSGSGTDFTLTISSLEPEDFAVYYCQQRSKYPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 448 aa, synthetic construct
QVELVESGGGVVQPGRSQRLSCAASGFTFSSHGMHWVRQAPGKGLEWVAIIAGDASTTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 215 aa, synthetic construct; identical to SEQ ID NO: 54

SEQ ID NO: 57 - protein, 448 aa, synthetic construct
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 215 aa, synthetic construct; identical to SEQ ID NO: 54

SEQ ID NO: 59 - protein, 448 aa, synthetic construct
QVELVESGGGVVQPGRSQRLSCAASGFTFSSYGMHWVRQAPGKGLEWVATIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 215 aa, synthetic construct; identical to SEQ ID NO: 54

SEQ ID NO: 61 - protein, 448 aa, synthetic construct
QVELVESGGGVVQPGRSQRLSCAASGFTFSSYGMHWVRQAPGKGLEWVAIINFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 215 aa, synthetic construct; identical to SEQ ID NO: 54

SEQ ID NO: 63 - protein, 448 aa, synthetic construct
QVELVESGGGVVQPGRSQRLSCAASGFTFSSAGMHWVRQAPGKGLEWVAIIWADGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 215 aa, synthetic construct; identical to SEQ ID NO: 54

SEQ ID NO: 65 - protein, 448 aa, synthetic construct
QVELVESGGGVVQPGRSQRLSCAASGFTFSSYGMHWVRQAPGKGLEWVAYIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 215 aa, synthetic construct; identical to SEQ ID NO: 54

SEQ ID NO: 67 - protein, 448 aa, synthetic construct
QVELVESGGGVVQPGRSQRLSCAASGFTFSSHGMHWVRQAPGKGLEWVATIWFDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 215 aa, synthetic construct
EIVLTQSPATLSLSPGERATLSCRASQSVSSTLAWYQQKPGQAPRLLIYDASKRATGIPARFSGSGSGTDFTLTISSLEPEDFAVYYCQQRSKYPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 448 aa, synthetic construct
QVELVESGGGVVQPGRSQRLSCAASGFTFSSYGMHWVRQAPGKGLEWVATIWHDGSSTYYADSVRGRFTISRDNSKNTLYLQMNSLRAEDTAVYFCARELGRRYFDLWGRGTLVSVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 215 aa, synthetic construct
EIVLTQSPATLSLSPGERATLSCRASQSVSSALAWYQQKPGQAPRLLIYDASKRATGIPARFSGSGSGTDFTLTISSLEPEDFAVYYCQQRGKYPPWTFGQGTKVESKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 5 aa, synthetic construct
  VARIANT 2: note = H, Y, A, or T
SXGMH

SEQ ID NO: 72 - protein, 17 aa, synthetic construct
  VARIANT 1: note = I, T, or Y
  VARIANT 3: note = W, N, or A
  VARIANT 4: note = F, H, A, or G
  VARIANT 6: note = G or A
  VARIANT 8: note = S or T
XIXXDXSXTYYADSVRG

SEQ ID NO: 73 - protein, 9 aa, synthetic construct
  VARIANT 3: note = G or N
ELXRRYFDL

SEQ ID NO: 74 - protein, 11 aa, synthetic construct
  VARIANT 9: note = Y, A, or T
RASQSVSSXLA

SEQ ID NO: 75 - protein, 10 aa, synthetic construct
  VARIANT 4: note = S or G
  VARIANT 6: note = Y or W
QQRXKXPPWT

(19) United States
(12) Patent Application Publication
(10) Pub. No.: US 2025/0197511 A1
(43) Pub. Date: Jun. 19, 2025

(54) METHODS FOR THE TREATMENT OF THYROID EYE DISEASE

(71) Applicant: HORIZON THERAPEUTICS IRELAND DAC, Dublin (IE)

(72) Inventors: Christopher Davidson WARD, Kensington, MD (US); Martin Jack BORROK, III, North Potomac, MD (US)

(73) Assignee: HORIZON THERAPEUTICS IRELAND DAC, Dublin (IE)

(21) Appl. No.: 18/978,929

(22) Filed: Dec. 12, 2024

Related U.S. Application Data
(60) Provisional application No. 63/609,824, filed on Dec. 13, 2023.

(51) Int. Cl. C07K 16/28; A61K 39/00

(52) U.S. Cl. CPC C07K 16/2863; A61K 2039/505; A61K 2039/54; C07K 2317/21; C07K 2317/52; C07K 2317/565; C07K 2317/732; C07K 2317/76; C07K 2317/92; C07K 2317/94

(57) ABSTRACT

Described herein are methods of treating or reducing the severity of thyroid eye disease (TED), as well as antibodies that bind and inhibit signaling through the insulin-like growth factor receptor 1, useful in the methods.

### CROSS-REFERENCE TO RELATED APPLICATIONS

This application claims the benefit of U.S. Provisional Application No. 63/609,824, filed Dec. 13, 2023, which is hereby incorporated by reference in its entirety.

### DESCRIPTION OF THE TEXT FILE SUBMITTED ELECTRONICALLY

The present application contains a Sequence Listing, which has been submitted electronically in XML format and is hereby incorporated by reference in its entirety. The computer readable format copy of the Sequence Listing, which was created on Dec. 10, 2024, is named 10853-US02-SEC_ST26.xml and is 73,099 bytes in size.

### FIELD OF THE INVENTION

The present invention relates to the fields of ophthalmology, endocrinology, and biopharmaceuticals. In particular, the present invention relates to methods for the treatment of thyroid eye disease by administering an antibody that inhibits the human insulin-like growth factor 1 receptor (IGF-1R).

### BACKGROUND OF THE INVENTION

Thyroid eye disease (TED), also known as thyroid-associated ophthalmopathy (TAO), Graves' ophthalmopathy or orbitopathy (GO), thyrotoxic exophthalmos, dysthyroid ophthalmopathy, and several other terms, is orbitopathy associated with thyroid dysfunction. TED can be classified into two types: active TED and inactive TED. Active TED, which typically lasts 1-3 years, is characterized by an ongoing autoimmune inflammatory response in the soft tissues of the orbit. Active TED is responsible for the expansion and remodeling of the ocular soft tissues. The inflammation of active TED spontaneously resolves and the condition transitions into inactive TED. Inactive TED is the term used to describe the long-term/permanent sequelae of active TED where orbital tissue expansion and remodeling persists. TED is typically associated with Graves' hyperthyroidism but can also occur as part of other autoimmune conditions that affect the thyroid gland and produce pathology in orbital and periorbital tissue, and, rarely, the pretibial skin (pretibial myxedema) or digits (thyroid acropachy). TED is an autoimmune orbitopathy in which the orbital and periocular soft tissues are primarily affected with secondary effects on the eye and vision. In TED, as a result of inflammation and expansion of orbital soft tissues, primarily eye muscles and adipose, the eyes are forced forward (bulge) out of their sockets—a phenomenon termed proptosis or exophthalmos.

A mounting body of evidence in the scientific literature indicates that the pathophysiology of active TED involves autoimmune activation and proliferation of orbital fibroblasts (Bahn, N Engl J Med., Vol. 362(8):726-738, 2010; Boschi et al., Br J Ophthalmol., Vol. 89(6):724-729, 2005; Smith, Pharmacol Rev., Vol. 62(2):199-236, 2010). The activation of fibroblasts triggers release of inflammatory cytokines, infiltration of immune cells into orbital soft tissues (muscle, interstitial and adipose), excessive synthesis of extracellular matrix, and tissue expansion and fibrotic remodeling. During the inactive phase, inflammation is absent and the disease plateaus, but significant remodeling of orbital tissue remains and rarely does the patient return to baseline.

The annual incidence rate of TED has been estimated at 16 cases per 100,000 women and 2.9 cases per 100,000 men from a study based in one largely rural Minnesota community. There appears to be a female preponderance in which women are affected 2.5-6 times more frequently than men; however, severe cases occur more often in men than in women. In addition, most patients are aged 30-50 years, with severe cases appearing to be more frequent in those older than 50 years. Although most cases of TED do not result in loss of vision, this condition can cause vision-threatening exposure keratopathy, troublesome diplopia (double vision), and compressive dysthyroid optic neuropathy.

Insulin-like growth factor-1 receptor (IGF-1R) is a tyrosine kinase cell surface receptor expressed on many tissues, including orbital fibroblasts. Signaling through this receptor plays a role in cell proliferation, differentiation, and inflammation. Inhibition of this receptor with an IGF-1R monoclonal antagonist antibody blocks the underlying immunopathogenesis that drives the orbital inflammation, excessive synthesis of extracellular matrix and tissue proliferation that are the hallmarks of TED (Pritchard et al., J Immunol., Vol. 170(12):6348-6354, 2003; Smith and Hoa, Clin Endocrinol Metab., Vol. 89(10):5076-5080, 2004; Hoa et al, PLoS One, Vol. 7(4):e34173, 2012). Teprotumumab is a fully human immunoglobulin G1 monoclonal antibody that binds with high affinity and selectivity to the extracellular domain of IGF-1R and prevents its activation by the endogenous ligands, IGF-1 and IGF-2. In phase 2 and 3 clinical trials in patients with moderate-to-severe TED, intravenously administered teprotumumab resulted in statistically significant and clinically relevant improvements in measures that assessed multiple facets of TED, including proptosis, inflammation as measured by Clinical Activity Score (CAS), diplopia, and quality of life. See Smith et al., N Engl J Med., Vol. 376(18):1748-1761, 2017; Douglas et al., N Engl J Med., Vol. 382(4):341-352, 2020; and Douglas et al., The Journal of Clinical Endocrinology & Metabolism, Vol. 109 (1): 25-35, 2024. Teprotumumab was approved in the United States for the treatment of thyroid eye disease in January 2020.

### SUMMARY OF THE INVENTION

The present invention is based, in part, on the design and generation of high affinity antibodies that specifically bind to and potently inhibit human IGF-1R. Such antibodies enable the use of lower doses and subcutaneous routes of administration leading to improved methods of treating thyroid eye disease. Subcutaneous administration of antibody treatments to a patient is a more convenient and cost-effective delivery route than intravenous administration since such treatments can be administered at home, and do not require the presence of a trained medical specialist. One hurdle to subcutaneous administration of antibodies, however, is the substantial reduction in volume of an antibody formulation to be able to be safely injected into the subcutaneous space and thus used in a pre-filled syringe, onbody infusor, autoinjector, etc. To address this challenge, high concentration antibody formulations are needed but this may be impractical for subcutaneous delivery due to viscosity challenges, aggregation behavior and other aggravating factors to formulate large molecules at high concentrations. One solution is the use of novel antibodies that bind a target with higher affinity and/or that exert higher biological activity than known antibodies. Additionally, antibodies comprising certain Fc region mutations possess longer half-life in vivo, which can further reduce the amount of antibody to be included in a subcutaneous formulation and reduce the frequency of administration.

Described herein are methods for treating thyroid eye disease (TED) using IGF-1R antibodies with high binding affinities and high biological inhibitory activity (e.g. IGF-1R antagonist antibodies).

Described herein in one aspect is a method of treating thyroid eye disease (TED) in an individual in need thereof, comprising administering to the individual an effective amount of an antibody or antigen binding fragment thereof that binds insulin-like growth factor 1 receptor (IGF-1R), wherein the antibody or antigen binding fragment thereof comprises: (a) an immunoglobulin heavy chain CDR1 (HCDR1) comprising the amino acid sequence SX₁GMH (SEQ ID NO: 71), wherein X₁ is H, Y, A, or T; (b) an immunoglobulin heavy chain CDR2 (HCDR2) comprising the amino acid sequence X₁IX₂X₃DX₄SX₅TYYADSVRG (SEQ ID NO: 72), wherein X₁ is I, T, or Y, X₂ is W, N, or A, X₃ is F, H, A, or G, X₄ is G or A, X₅ is S or T; (c) an immunoglobulin heavy chain CDR3 (HCDR3) comprising the amino acid sequence ELX₁RRYFDL (SEQ ID NO: 73), wherein X₁ is G or N; (d) an immunoglobulin light chain CDR1 (LCDR1) comprising the amino acid sequence RASQSVSSX₁LA (SEQ ID NO: 74), wherein X₁ is Y, A, or T; (e) an immunoglobulin light chain CDR2 (LCDR2) comprising the amino acid sequence DASKRAT (SEQ ID NO: 46); and (f) an immunoglobulin light chain CDR3 (LCDR3) comprising the amino acid sequence QQRX₁KX₂PPWT (SEQ ID NO: 75), wherein X₁ is S or G, X₂ is Y or W; wherein the antibody or antigen binding fragment thereof does not comprise an immunoglobulin heavy chain variable region identical to SEQ ID NO: 1 and/or an immunoglobulin light chain variable region identical to SEQ ID NO: 2, thereby treating the TED. In some cases, the amino acid residue corresponding to X₂ of the LCDR3 is a tyrosine.

In certain embodiments, an IGF-1R antagonist antibody is administered to a patient in need of treatment of thyroid eye disease, wherein the IGF-1R antagonist antibody comprises an immunoglobulin heavy chain variable region comprising an HCDR1, an HCDR2, and an HCDR3 and an immunoglobulin light chain variable region comprising an LCDR1, an LCDR2, and an LCDR3, wherein (a) HCDR1, HCDR2 and HCDR3 comprise the amino acid sequences of SEQ ID NOs: 30, 35, and 41, respectively, and LCDR1, LCDR2, and LCDR3 comprise the amino acid sequences of SEQ ID NOs: 43, 46, and 48, respectively; (b) HCDR1, HCDR2 and HCDR3 comprise the amino acid sequences of SEQ ID NOs: 33, 38, and 41, respectively, and LCDR1, LCDR2, and LCDR3 comprise the amino acid sequences of SEQ ID NOs: 45, 46, and 48, respectively; (c) HCDR1, HCDR2 and HCDR3 comprise the amino acid sequences of SEQ ID NOs: 30, 34, and 41, respectively, and LCDR1, LCDR2, and LCDR3 comprise the amino acid sequences of SEQ ID NOs: 45, 46, and 48, respectively; (d) HCDR1, HCDR2 and HCDR3 comprise the amino acid sequences of SEQ ID NOs: 31, 35, and 42, respectively, and LCDR1, LCDR2, and LCDR3 comprise the amino acid sequences of SEQ ID NOs: 45, 46, and 48, respectively; (e) HCDR1, HCDR2 and HCDR3 comprise the amino acid sequences of SEQ ID NOs: 33, 40, and 41, respectively, and LCDR1, LCDR2, and LCDR3 comprise the amino acid sequences of SEQ ID NOs: 45, 46, and 48, respectively; (f) HCDR1, HCDR2 and HCDR3 comprise the amino acid sequences of SEQ ID NOs: 33, 37, and 41, respectively, and LCDR1, LCDR2, and LCDR3 comprise the amino acid sequences of SEQ ID NOs: 45, 46, and 48, respectively; (g) HCDR1, HCDR2 and HCDR3 comprise the amino acid sequences of SEQ ID NOs: 32, 36, and 41, respectively, and LCDR1, LCDR2, and LCDR3 comprise the amino acid sequences of SEQ ID NOs: 45, 46, and 48, respectively; (h) HCDR1, HCDR2 and HCDR3 comprise the amino acid sequences of SEQ ID NOs: 33, 35, and 41, respectively, and LCDR1, LCDR2, and LCDR3 comprise the amino acid sequences of SEQ ID NOs: 45, 46, and 48, respectively; (i) HCDR1, HCDR2 and HCDR3 comprise the amino acid sequences of SEQ ID NOs: 30, 40, and 41, respectively, and LCDR1, LCDR2, and LCDR3 comprise the amino acid sequences of SEQ ID NOs: 44, 46, and 48, respectively; or (j) HCDR1, HCDR2 and HCDR3 comprise the amino acid sequences of SEQ ID NOs: 33, 39, and 41, respectively, and LCDR1, LCDR2, and LCDR3 comprise the amino acid sequences of SEQ ID NOs: 43, 46, and 47, respectively.

In some embodiments of the methods of the invention, the HCDR1 comprises the amino acid sequence of SEQ ID NO: 30 (SHGMH), the HCDR2 comprises the amino acid sequence of SEQ ID NO: 35 (YIWFDGSSTYYADSVRG), the HCDR3 comprises the amino acid sequence of SEQ ID NO: 41 (ELGRRYFDL), the LCDR1 comprises the amino acid sequence of SEQ ID NO: 43 (RASQSVSSALA), the LCDR2 comprises the amino acid sequence of SEQ ID NO: 46 (DASKRAT), and the LCDR3 comprises the amino acid sequence of SEQ ID NO: 48 (QQRSKYPPWT).

In some embodiments of the methods of the invention, the HCDR1 comprises the amino acid sequence of SEQ ID NO: 33 (SYGMH), the HCDR2 comprises the amino acid sequence of SEQ ID NO: 38 (IIWFDGSSTYYADSVRG), the HCDR3 comprises the amino acid sequence of SEQ ID NO: 41 (ELGRRYFDL), the LCDR1 comprises the amino acid sequence of SEQ ID NO: 45 (RASQSVSSYLA), the LCDR2 comprises the amino acid sequence of SEQ ID NO: 46 (DASKRAT), and the LCDR3 comprises the amino acid sequence of SEQ ID NO: 48 (QQRSKYPPWT).

In some embodiments of the methods of the invention, the HCDR1 comprises the amino acid sequence of SEQ ID NO: 30 (SHGMH), the HCDR2 comprises the amino acid sequence of SEQ ID NO: 34 (IIAGDASTTYYADSVRG), the HCDR3 comprises the amino acid sequence of SEQ ID NO: 41 (ELGRRYFDL), the LCDR1 comprises the amino acid sequence of SEQ ID NO: 45 (RASQSVSSYLA), the LCDR2 comprises the amino acid sequence of SEQ ID NO: 46 (DASKRAT), and the LCDR3 comprises the amino acid sequence of SEQ ID NO: 48 (QQRSKYPPWT).

In certain embodiments of the methods of the invention, the IGF-1R antagonist antibody or antigen binding fragment thereof comprises an immunoglobulin heavy chain variable region that comprises an amino acid sequence that is at least 90% identical to an amino acid sequence selected from SEQ ID NOs: 3, 5, 7, 9, 11, 13, 15, 17, 19, and 21. In these and other embodiments, the IGF-1R antagonist antibody or antigen binding fragment thereof comprises an immunoglobulin light chain variable region that comprises an amino acid sequence that is at least 90% identical to an amino acid sequence selected from SEQ ID NOs: 4, 6, 8, 10, 12, 14, 16, 18, 20, and 22. For instance, in some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 3; and wherein the immunoglobulin light chain variable region comprises an amino acid sequence that is at least about 90%, 95%, 97%, 99%, or 100% identical to that set forth in SEQ ID NO: 4.

In some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 5; and wherein the immunoglobulin light chain variable region comprises an amino acid sequence that is at least about 90%, 95%, 97%, 99%, or 100% identical to that set forth in SEQ ID NO: 6.

In some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 7; and wherein the immunoglobulin light chain variable region comprises an amino acid sequence that is at least about 90%, 95%, 97%, 99%, or 100% identical to that set forth in SEQ ID NO: 8.

In some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 9; and wherein the immunoglobulin light chain variable region comprises an amino acid sequence that is at least about 90%, 95%, 97%, 99%, or 100% identical to that set forth in SEQ ID NO: 10.

In some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 11; and wherein the immunoglobulin light chain variable region comprises an amino acid sequence that is at least about 90%, 95%, 97%, 99%, or 100% identical to that set forth in SEQ ID NO: 12.

In some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 13; and wherein the immunoglobulin light chain variable region comprises an amino acid sequence that is at least about 90%, 95%, 97%, 99%, or 100% identical to that set forth in SEQ ID NO: 14.

In some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 15; and wherein the immunoglobulin light chain variable region comprises an amino acid sequence that is at least about 90%, 95%, 97%, 99%, or 100% identical to that set forth in SEQ ID NO: 16.

In some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 17; and wherein the immunoglobulin light chain variable region comprises an amino acid sequence that is at least about 90%, 95%, 97%, 99%, or 100% identical to that set forth in SEQ ID NO: 18.

In some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 19; and wherein the immunoglobulin light chain variable region comprises an amino acid sequence that is at least about 90%, 95%, 97%, 99%, or 100% identical to that set forth in SEQ ID NO: 20.

In some embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 21; and wherein the immunoglobulin light chain variable region comprises an amino acid sequence that is at least about 90%, 95%, 97%, 99%, or 100% identical to that set forth in SEQ ID NO: 22.

In certain embodiments of the methods of the invention, the IGF-1R antagonist antibody comprises an immunoglobulin heavy chain comprising an amino acid sequence that is at least 90% identical to an amino acid sequence selected from SEQ ID NOs: 51, 53, 55, 57, 59, 61, 63, 65, 67, and 69, and an immunoglobulin light chain comprising an amino acid sequence that is at least 90% identical to an amino acid sequence selected from SEQ ID NOs: 52, 54, 56, 58, 60, 62, 64, 66, 68, and 70. For instance, in some embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 51; and wherein the immunoglobulin light chain comprises an amino acid sequence that is at least about 90%, 95%, 97%, 99%, or 100% identical to that set forth in SEQ ID NO: 52.

In some embodiments of the methods of the invention, the IGF-1R antagonist antibody may comprise: (a) an immunoglobulin heavy chain comprising the amino acid sequence of SEQ ID NO: 51 and an immunoglobulin light chain comprising the amino acid sequence of SEQ ID NO: 52; (b) an immunoglobulin heavy chain comprising the amino acid sequence of SEQ ID NO: 57 and an immunoglobulin light chain comprising the amino acid sequence of SEQ ID NO: 58; (c) an immunoglobulin heavy chain comprising the amino acid sequence of SEQ ID NO: 55 and an immunoglobulin light chain comprising the amino acid sequence of SEQ ID NO: 56; (d) an immunoglobulin heavy chain comprising the amino acid sequence of SEQ ID NO: 53 and an immunoglobulin light chain comprising the amino acid sequence of SEQ ID NO: 54; (e) an immunoglobulin heavy chain comprising the amino acid sequence of SEQ ID NO: 59 and an immunoglobulin light chain comprising the amino acid sequence of SEQ ID NO: 60; (f) an immunoglobulin heavy chain comprising the amino acid sequence of SEQ ID NO: 61 and an immunoglobulin light chain comprising the amino acid sequence of SEQ ID NO: 62; (g) an immunoglobulin heavy chain comprising the amino acid sequence of SEQ ID NO: 63 and an immunoglobulin light chain comprising the amino acid sequence of SEQ ID NO: 64; (h) an immunoglobulin heavy chain comprising the amino acid sequence of SEQ ID NO: 65 and an immunoglobulin light chain comprising the amino acid sequence of SEQ ID NO: 66; (i) an immunoglobulin heavy chain comprising the amino acid sequence of SEQ ID NO: 67 and an immunoglobulin light chain comprising the amino acid sequence of SEQ ID NO: 68; or (j) an immunoglobulin heavy chain comprising the amino acid sequence of SEQ ID NO: 69 and an immunoglobulin light chain comprising the amino acid sequence of SEQ ID NO: 70.

In certain embodiments, the IGF-1R antagonist antibody or antigen binding fragment thereof administered according to the methods of the invention is a monoclonal antibody or antigen binding fragment thereof. In some aspects of the methods of the invention, the IGF-1R antagonist antibody or antigen binding fragment thereof is an IgG antibody or antigen binding fragment thereof. In some aspects, the antigen binding fragment of an IGF-1R antagonist antibody comprises a Fab, F(ab)₂, or a single chain variable fragment (scFv). In some aspects, the IGF-1R antagonist antibody or antigen binding fragment thereof is chimeric or humanized. In certain other embodiments, the IGF-1R antagonist antibody or antigen binding fragment thereof administered according to the methods of the invention is a human antibody or antigen binding fragment thereof. In certain embodiments, the IGF-1R antagonist antibody is a full-length antibody.

In some embodiments of the methods of the invention, the IGF-1R antagonist antibody comprises a constant region from a human IgG immunoglobulin, such as a human IgG1 immunoglobulin. In some such embodiments, the IGF-1R antagonist antibody comprises a M252Y, a S254T, and a T256E substitution according to EU numbering in one or both heavy chain constant regions.

In some cases, the IGF-1R antagonist antibody or antigen binding fragment thereof is characterized by a half-life of 25 days or longer in a human.

In some cases, the IGF-1R antagonist antibody or antigen binding fragment thereof is characterized by a half-life of 30 days or longer in a human.

In some cases, the IGF-1R antagonist antibody inhibits signaling through IGF-1R. In some cases, the IGF-1R antagonist antibody inhibits phosphorylation of IGF-1R with an EC50 of 10 ng/mL or less. In some cases, the IGF-1R antagonist antibody inhibits phosphorylation of IGF-1R with an EC50 of 9 ng/mL or less. In some cases, the IGF-1R antagonist antibody inhibits phosphorylation of IGF-1R with an EC50 of 7 ng/mL or less. In some cases, the IGF-1R antagonist antibody binds to IGF-1R with a K_{D} of less than 5×10⁻⁹ M. In some cases, the IGF-1R antagonist antibody binds to IGF-1R with a K_{D} of less than 1×10⁻⁹ M. In some cases, the IGF-1R antagonist antibody binds to IGF-1R with a K_{D} of less than 5×10⁻¹⁰ M.

In certain embodiments of the methods of the invention, the IGF-1R antagonist antibody or antigen binding fragment thereof is administered to the individual or patient with thyroid eye disease intravenously or is formulated for intravenous administration. In certain other embodiments of the methods of the invention, the IGF-1R antagonist antibody or antigen binding fragment thereof is administered to the individual or patient with thyroid eye disease subcutaneously (e.g. by subcutaneous injection) or is formulated for subcutaneous administration.

Patients or individuals to be treated according to the methods of the invention may, in some embodiments, have or be diagnosed with moderate to severe TED. In such embodiments, the patient or individual may have one or more of the following: lid retraction ≥2 mm, moderate or severe soft tissue involvement, proptosis ≥3 mm above normal for race and gender, and inconstant or constant diplopia (Gorman score 2-3). In some such embodiments, the patient or individual has diplopia (intermittent, inconstant, or constant diplopia; Gorman score 1-3) prior to administration of the IGF-1R antagonist antibody. In further embodiments, the patient or individual has an increase in proptosis of 3 mm or more in at least one eye prior to administration of the IGF-1R antagonist antibody as compared to the normal average for race and gender of the patient or individual.

In some embodiments, patients or individuals to be treated according to the methods of the invention may have or be diagnosed with active thyroid eye disease. In some such embodiments, the patient or individual may have a clinical activity score (CAS) of 3 or more on the 7-component scale or a CAS of 4 or more on the 10-component scale in at least one eye. In other embodiments, patients or individuals to be treated according to the methods of the invention may have or be diagnosed with inactive thyroid eye disease. In such embodiments, the patient or individual may have a CAS no more than 2 on the 7-component scale or 3 on the 10-component scale in either eye. In certain embodiments, a patient or individual with inactive thyroid eye disease may have diplopia, an increase in proptosis, or restricted eye motility in any direction of gaze.

In some embodiments, the methods of the invention reduce proptosis by at least 2 mm in the individual or patient with thyroid eye disease. In other embodiments, proptosis is reduced by at least 3 mm. In still other embodiments, proptosis is reduced by at least 4 mm.

In some embodiments, the methods of the invention reduce the CAS of the individual or patient with thyroid eye disease. In some embodiments, the CAS is reduced by at least 2 points. In other embodiments, the CAS is reduced by at least 3 points. In yet other embodiments, the CAS of the individual in need thereof is reduced to one or less. In certain embodiments, the CAS of the individual in need thereof is reduced to zero.

In certain embodiments, the methods of the invention reduce the severity of diplopia in the individual or patient with thyroid eye disease. In some embodiments, the diplopia is constant diplopia. In other embodiments, the diplopia is intermittent diplopia. In still other embodiments, the diplopia is inconstant diplopia. In some embodiments, the reduction in the severity of diplopia is sustained for at least 20 weeks after discontinuation of the IGF-1R antagonist antibody or antigen binding fragment thereof. In some embodiments, the improvement in or reduction in severity of diplopia is sustained for at least 50 weeks after discontinuation of the IGF-1R antagonist antibody or antigen binding fragment thereof.

In some embodiments, the present invention includes methods for improving quality of life in the individual or patient with thyroid eye disease comprising administering to the individual or patient an IGF-1R antagonist antibody or antigen binding fragment as described herein. In some cases, the quality of life is measured by the Graves' Ophthalmopathy Quality of Life (GO-QoL) assessment, on either the visual functioning subscale or appearance subscale thereof. In some cases, the GO-QoL is improved by at least 8 points. In some cases, visual function in the patient or individual is improved as measured by the visual functioning subscale. In some cases, the appearance of the individual or patient is improved as measured by the appearance subscale.

The use of IGF-1R antagonist antibodies or antigen binding fragments thereof in any of the methods disclosed herein or for preparation of medicaments for administration according to any of the methods disclosed herein is specifically contemplated. For instance, the present invention includes an IGF-1R antagonist antibody or antigen binding fragment thereof for use in a method of treating thyroid eye disease in a patient or individual in need thereof, wherein the method comprises administering to the patient or individual any of the IGF-1R antagonist antibodies or antigen binding fragments thereof described herein. The present invention also encompasses the use of any of the IGF-1R antagonist antibodies or antigen binding fragments thereof described herein for preparation of a medicament for treating thyroid eye disease in a patient or individual in need thereof.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A illustrates multiple sequence alignment of heavy chain variable regions described herein. Heavy chain variable regions for teprotumumab, B02, B09, E01, B10, D10, C10, A11, D03, F02, and H11 are set forth in SEQ ID NOs: 1, 3, 5, 7, 9, 11, 13, 15, 17, 19, and 21, respectively.

FIG. 1B illustrates multiple sequence alignment of light chain variable regions described herein. Light chain variable regions for teprotumumab, B02, B09, E01, B10, D10, C10, A11, D03, F02, and H11 are set forth in SEQ ID NOs: 2, 4, 6, 8, 10, 12, 14, 16, 18, 20, and 22, respectively.

FIG. 2 illustrates an antibody dependent cell cytotoxicity (ADCC) assay performed with clone D03 formatted with different heavy chain constant regions (the “YTE” mutation, which includes mutations at M252Y/S254T/T256E according to EU numbering; and the “LS” mutation which includes mutations at Met428Leu/Asn434Ser according to EU numbering). RLU=Relative light units.

FIG. 3 illustrates inhibition of IGF-1R signaling by teprotumumab and D03-YTE.

### DETAILED DESCRIPTION

In the following description, certain specific details are set forth in order to provide a thorough understanding of various embodiments. However, one skilled in the art will understand that the embodiments provided may be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

As used herein the term “about” refers to an amount that is near the stated amount by 10% or less.

As used herein the terms “individual,” “patient,” or “subject” are used interchangeably and refer to individuals diagnosed with, suspected of being afflicted with, or at-risk of developing at least one disease for which the described compositions and methods are useful for treating (e.g. thyroid eye disease). In certain embodiments, the individual is a mammal. In certain embodiments, the mammal is a mouse, rat, rabbit, dog, cat, horse, cow, sheep, pig, goat, llama, alpaca, or yak. In certain embodiments, the individual is a human.

Among the provided antibodies are monoclonal antibodies, multispecific antibodies (for example, bispecific antibodies and polyreactive antibodies), and antibody fragments. The antibodies include antibody-conjugates and molecules comprising the antibodies, such as chimeric molecules. Thus, an antibody includes, but is not limited to, full-length and native antibodies, as well as fragments and portions thereof retaining the binding specificities thereof, such as any specific binding portion thereof including those having any number of, immunoglobulin classes and/or isotypes (e.g., IgG1, IgG2, IgG3, IgG4, IgA, IgD, IgE and IgM); and biologically relevant (antigen-binding) fragments or specific binding portions thereof, including but not limited to Fab, F(ab′)₂, Fv, and scFv (single chain or related entity). A monoclonal antibody is generally one within a composition of substantially homogeneous antibodies; thus, any individual antibodies comprised within the monoclonal antibody composition are identical except for possible naturally occurring mutations that may be present in minor amounts. The monoclonal antibody can comprise a human IgG1 constant region. The monoclonal antibody can comprise a human IgG2 constant region. The monoclonal antibody can comprise a human IgG4 constant region. In some embodiments, the monoclonal antibody can have engineered constant regions that comprise domains from more than on isotype. For instance, the monoclonal antibody may have a constant region that comprises domains from a human IgG1 constant region and a human IgG4 constant region. In some embodiments, the monoclonal antibody may have a constant region that comprises domains from a human IgG2 constant region and a human IgG4 constant region.

The term “antibody” herein is used in the broadest sense and includes monoclonal antibodies, and includes intact antibodies and functional (antigen-binding) antibody fragments thereof. The term encompasses genetically engineered and/or otherwise modified forms of immunoglobulins, such as chimeric antibodies, fully human antibodies, humanized antibodies, and heteroconjugate antibodies, multispecific, e.g., bispecific, antibodies. The term also encompasses intact or full-length antibodies, including antibodies of any class or sub-class, including IgG and sub-classes thereof, IgM, IgE, IgA, and IgD. The antibody can comprise a human IgG1 constant region. The antibody can comprise a human IgG4 constant region. The antibody can comprise a human IgG2 constant region. In some embodiments, the antibody may comprise constant regions comprised of domains from two different human IgG constant regions, such as a constant region comprised of domains from both human IgG1 and human IgG4 (e.g. IgG1/IgG4 chimeric constant region) or a constant region comprised of domains from both human IgG2 and human IgG4 (e.g. IgG2/IgG4 chimeric constant region). An intact or full-length antibody refers to a tetrameric immunoglobulin protein comprising two light chain polypeptides (about 25 kDa each) and two heavy chain polypeptides (about 50-70 kDa each). The term “light chain” or “immunoglobulin light chain” refers to a polypeptide comprising, from amino terminus to carboxyl terminus, a single immunoglobulin light chain variable region (VL) and a single immunoglobulin light chain constant domain (CL). The immunoglobulin light chain constant domain (CL) can be a human kappa (κ) or human lambda (λ) constant domain. The term “heavy chain” or “immunoglobulin heavy chain” refers to a polypeptide comprising, from amino terminus to carboxyl terminus, a single immunoglobulin heavy chain variable region (VH), an immunoglobulin heavy chain constant domain 1 (CH1), an immunoglobulin hinge region, an immunoglobulin heavy chain constant domain 2 (CH2), an immunoglobulin heavy chain constant domain 3 (CH3), and optionally an immunoglobulin heavy chain constant domain 4 (CH4). Heavy chains are classified as mu (μ), delta (Δ), gamma (γ), alpha (α), and epsilon (ε), and define the antibody's isotype as IgM, IgD, IgG, IgA, and IgE, respectively. The heavy chains in IgG, IgA, and IgD antibodies have three constant domains (CH1, CH2, and CH3), whereas the heavy chains in IgM and IgE antibodies have four constant domains (CH1, CH2, CH3, and CH4). The immunoglobulin heavy chain constant domains can be from any immunoglobulin isotype, including subtypes. The antibody chains are linked together via inter-polypeptide disulfide bonds between the CL domain and the CH1 domain (i.e. between the light and heavy chain) and between the hinge regions of the two antibody heavy chains. In certain embodiments, the IGF-1R antagonist antibodies administered according to the methods of the invention are full-length antibodies.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

The term “variable region” or “variable domain” refers to the domain of an antibody heavy or light chain that is involved in binding the antibody to antigen. The variable domains of the heavy chain and light chain (VH and V_{L}, respectively) of a native antibody generally have similar structures, with each domain comprising four conserved framework regions (FRs) and three CDRs (See e.g., Kindt et al. Kuby Immunology, 6th ed., W.H. Freeman and Co., page 91(2007)). A single VH or V_{L} domain may be sufficient to confer antigen-binding specificity. Furthermore, antibodies that bind a particular antigen may be isolated using a VH or V_{L} domain from an antibody that binds the antigen to screen a library of complementary V_{L} or VH domains, respectively (See e.g., Portolano et al., J. Immunol. 150:880-887 (1993); Clarkson et al., Nature 352:624-628 (1991)).

Specific binding or binding of antibody molecules described herein refers to binding mediated by one or more CDR portions of the antibody. Not all CDRs may be required for specific binding. Specific binding can be demonstrated for example by an ELISA against a specific recited target or antigen that shows significant increase in binding compared to an isotype control antibody.

As described herein an “epitope” refers to the binding determinant of an antibody or fragment described herein minimally necessary for specific binding of the antibody or fragment thereof to a target antigen. When the target antigen is a polypeptide the epitope will be a continuous or discontinuous epitope. A continuous epitope is formed by one region of the target antigen, while a discontinuous epitope may be formed from two or more separate regions. A discontinuous epitope, for example, may form when a target antigen adopts a tertiary structure that brings two amino acid sequences together and forms a three-dimensional structure bound by the antibody. When the target antigen is a polypeptide the epitope will generally be a plurality of amino acids linked into a polypeptide chain. A continuous epitope may comprise 5, 6, 7, 8, 9, 10, 11, 12, 13, 14, 15, 16, 17, 18, 19, or 20 contiguous amino acids. While an epitope may comprise a contiguous polymer of amino acids, not every amino acid of the polymer may be contacted by an amino acid residue of the antibody. Such non-contacted amino acids will still comprise part of the epitope as they may be important for the structure and linkage of the contacted amino acids. The skilled artisan may determine if any given antibody binds an epitope of a reference antibody, for example, by cross-blocking experiments with a reference antibody. In certain embodiments, described herein, are antibodies that bind the same epitope of the described antibodies. In certain embodiments, described herein, are antibodies that are competitively blocked by the described antibodies. In certain embodiments, described herein, are antibodies that compete for binding with the described antibodies.

Among the provided antibodies are antibody fragments. An “antibody fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′)₂; diabodies; linear antibodies; single-chain antibody molecules (e.g. scFv or sFv); and multispecific antibodies formed from antibody fragments. In particular embodiments, the antibody fragments are single-chain antibody fragments comprising a variable heavy chain region and/or a variable light chain region, such as scFvs.

Antibody fragments can be made by various techniques, including but not limited to proteolytic digestion of an intact antibody as well as production by recombinant host cells. In some embodiments, the antibody fragments are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A “humanized” antibody is an antibody in which all or substantially all CDR amino acid residues are derived from non-human CDRs and all or substantially all FR amino acid residues are derived from human FRs. A humanized antibody optionally may include at least a portion of an antibody constant region derived from a human antibody. A “humanized form” of a non-human antibody refers to a variant of the non-human antibody that has undergone humanization, typically to reduce immunogenicity to humans, while retaining the specificity and affinity of the parental non-human antibody. In some embodiments, some FR residues in a humanized antibody are substituted with corresponding residues from a non-human antibody (e.g., the antibody from which the CDR residues are derived), e.g., to restore or improve antibody specificity or affinity.

Among the provided antibodies are human antibodies. A “human antibody” is an antibody with amino acid sequences corresponding to or derived from human germ line immunoglobulin sequences, such as that of an antibody produced by a human or a human cell, or non-human source that utilizes human antibody repertoires or other human antibody-encoding sequences, including human antibody libraries. The term excludes humanized forms of non-human antibodies comprising non-human antigen-binding regions, such as those in which all or substantially all CDRs are non-human.

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Human antibodies also may be derived from human antibody libraries, including phage display and cell-free libraries, containing antibody-encoding sequences derived from a human repertoire.

The terms “polypeptide” and “protein” are used interchangeably to refer to a polymer of amino acid residues, and are not limited to a minimum length. Polypeptides, including the provided antibodies and antibody chains and other peptides, e.g., linkers and binding peptides, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification. In some embodiments, amino acid sequence variants of the antibodies provided herein are contemplated. A variant typically differs from a polypeptide specifically disclosed herein in one or more substitutions, deletions, additions and/or insertions. Such variants can be naturally occurring or can be synthetically generated, for example, by modifying one or more of the polypeptide sequences of the invention and evaluating one or more biological activities of the polypeptide as described herein and/or using any of a number of known techniques. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Affinity of an antibody for its target can be measured by the dissociation constant (K_{D}). In some embodiments, an antibody provided herein has a dissociation constant (K_{D}) of about 1 μM, 100 nM, 50 nM, 40 nM, 30 nM, 20 nM, 10 nM, 5 nM, 2 nM, 1 nM, 0.5 nM, 0.1 nM, 0.05 nM, 0.01 nM or less (e.g., 10⁻⁸ M or less, e.g., from 10⁻⁸ M to 10⁻¹³ M, e.g., from 10⁻⁹ M to 10⁻¹³ M) for the antibody target (e.g. IGF-1R). In some embodiments, an antibody provided herein has a dissociation constant (K_{D}) of about 100 nM, 50 nM, 40 nM, 30 nM, 20 nM, 10 nM, 5 nM, 2 nM, 1 nM, 0.5 nM, 0.1 nM, 0.05 nM, 0.01 nM, or 0.001 nM or greater (e.g., 10⁻⁸ M or less, e.g., from 10⁻⁸ M to 10⁻¹³ M, e.g., from 10⁻⁹ M to 10⁻¹³ M) for the antibody target (e.g. IGF-1R). Antibody affinity and the associated K_{D} can be measured by any suitable assay, including but not limited to, surface plasmon resonance assays, Kinetic Exclusion Assays (KinExA), and bio-layer interferometry assays. In certain embodiments, affinity of the antibodies and associated K_{D} can be measured using surface plasmon resonance assays (e.g., using a BIACORE®-2000 or a BIACORE®-3000). In other embodiments, affinity of the antibodies and associated K_{D} are measured by a bio-layer interferometry method, such as that described in Kumaraswamy et al., Methods Mol. Biol., Vol. 1278:165-82, 2015 and employed in Octet® systems (Pall ForteBio).

In some embodiments, one or more amino acid modifications may be introduced into the Fc region of an antibody provided herein, thereby generating an Fc region variant. An Fc region, as the term is used herein, is a C-terminal region of an immunoglobulin heavy chain that contains at least a portion of the constant region. The term “constant region,” used interchangeably herein with “constant domain,” refers to all domains of an antibody other than the variable region. The constant region is not involved directly in binding of an antigen but exhibits various effector functions. The Fc region of an immunoglobulin generally comprises two constant domains, a CH2 domain and a CH3 domain, and optionally comprises a CH4 domain. An Fc region includes native sequence Fc regions and variant Fc regions. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the Fc region of an immunoglobulin is important for many important antibody functions (e.g. effector functions), such as antibody-dependent cellular cytotoxicity (ADCC), complement dependent cytotoxicity (CDC), and antibody-dependent cell-mediated phagocytosis (ADCP), all of which result in killing of target cells, albeit by different mechanisms. Accordingly, in some embodiments, the antibodies described herein comprise the variable domains described herein combined with immunoglobulin constant domains (e.g. human immunoglobulin constant domains) comprising different Fc regions, selected based on the biological activities of the antibody for the intended use. In certain instances, human IgGs, for example, can be classified into four subclasses, IgG1, IgG2, IgG3, and IgG4, and each these of these comprises an Fc region having a unique profile for binding to one or more of Fcγ receptors (activating receptors FcγRI (CD64), FcγRIIA, FcγRIIC (CD32); FcγRIIIA and FcγRIIIB (CD16) and inhibiting receptor FcγRIIB), and for the first component of complement (C1q). Human IgG1 and IgG3 bind to all Fcγ receptors; IgG2 binds to FcγRIIA_{H131}, and with lower affinity to FcγRIIA_{R131} FcγRIIIA_{V158}; IgG4 binds to FcγRI, FcγRIIA, FcγRIIB, FcγRIIC, and FcγRIIIA_{V158}; and the inhibitory receptor FcγRIIB has a lower affinity for IgG1, IgG2 and IgG3 than all other Fcγ receptors. Studies have shown that FcγRI does not bind to IgG2, and FcγRIIIB does not bind to IgG2 or IgG4. In general, with regard to ADCC activity, human IgG1≥IgG3IgG4≥IgG2.

In some embodiments, the antibodies used according to the methods of the invention are variants that possess effector functions, which make it a desirable candidate for applications in which certain effector functions (such as complement fixation and ADCC) are unnecessary or deleterious. Such antibodies can have decreased complement-dependent cytotoxicity (CDC), antibody-dependent cell cytotoxicity (ADCC), or antibody dependent cellular phagocytosis (ADCP). In other embodiments, the antibodies of this disclosure are variants that possess increased effector functions for applications in which increased effector function would be beneficial. Such antibodies can have increased CDC, ADCC, or ADCP, or a combination thereof. Non-limiting examples of in vitro assays to assess ADCC activity of a molecule of interest is described in U.S. Pat. Nos. 5,500,362 and 5,821,337. Alternatively, non-radioactive assays methods may be employed (e.g., ACTI™ and CytoTox 96® non-radioactive cytotoxicity assays). Useful effector cells for such assays include peripheral blood mononuclear cells (PBMC), monocytes, macrophages, and Natural Killer (NK) cells.

Antibodies can have increased half-lives and improved binding to the neonatal Fc receptor (FcRn) (See e.g., US 2005/0014934). Such antibodies can comprise an Fc region with one or more substitutions therein which improve binding of the Fc region to FcRn, and include those with substitutions at one or more of the following Fc region residues: 238, 256, 265, 272, 286, 303, 305, 307, 311, 312, 317, 340, 356, 360, 362, 376, 378, 380, 382, 413, 424 or 434 according to the EU numbering system (See e.g., U.S. Pat. No. 7,371,826). Other examples of Fc region variants are also contemplated (See e.g., Duncan & Winter, Nature 322:738-40 (1988); U.S. Pat. Nos. 5,648,260 and 5,624,821; and WO94/29351). One such set of mutations that confers increased half-life is the “YTE” mutation, which includes mutations at M252Y, S254T, and T256E according to EU numbering in a heavy chain constant region. Another such set of mutations that has been reported to confer increased half-life is the “LS” mutation, which includes mutations at Met428Leu and Asn434Ser according to EU numbering in a heavy chain constant region.

In some embodiments, it may be desirable to create cysteine engineered antibodies, e.g., “thioMAbs,” in which one or more residues of an antibody are substituted with cysteine residues. In some embodiments, the substituted residues occur at accessible sites of the antibody. Reactive thiol groups can be positioned at sites for conjugation to other moieties, such as drug moieties or linker drug moieties, to create an immunoconjugate. In some embodiments, any one or more of the following residues may be substituted with cysteine: V205 (Kabat numbering) of the light chain; A118 (EU numbering) of the heavy chain; and S400 (EU numbering) of the heavy chain Fc region.

In some embodiments, an antibody provided herein may be further modified to contain additional nonproteinaceous moieties that are known and available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n vinyl pyrrolidone)polyethylene glycol, polypropylene glycol homopolymers, polypropylen oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if two or more polymers are attached, they can be the same or different molecules.

The antibodies described herein can be encoded by a nucleic acid. A nucleic acid is a type of polynucleotide comprising two or more nucleotide bases. In certain embodiments, the nucleic acid is a component of a vector that can be used to transfer the polypeptide encoding polynucleotide into a cell. As used herein, the term “vector” refers to a nucleic acid molecule capable of transporting another nucleic acid to which it has been linked. One type of vector is a genomic integrated vector, or “integrated vector,” which can become integrated into the chromosomal DNA of the host cell. Another type of vector is an “episomal” vector, e.g., a nucleic acid capable of extra-chromosomal replication. Vectors capable of directing the expression of genes to which they are operatively linked are referred to herein as “expression vectors.” Suitable vectors comprise plasmids, bacterial artificial chromosomes, yeast artificial chromosomes, viral vectors and the like. In the expression vectors, regulatory elements such as promoters, enhancers, polyadenylation signals for use in controlling transcription can be derived from mammalian, microbial, viral or insect genes. The ability to replicate in a host, usually conferred by an origin of replication, and a selection gene to facilitate recognition of transformants may additionally be incorporated. Vectors derived from viruses, such as lentiviruses, retroviruses, adenoviruses, adeno-associated viruses, and the like, may be employed. Plasmid vectors can be linearized for integration into a genomic region. In certain embodiments, the expression vector is a plasmid. In certain embodiments, the expression vector is a lentivirus, adenovirus, or adeno-associated virus. In certain embodiments, the expression vector is an adenovirus. In certain embodiments, the expression vector is an adeno-associated virus. In certain embodiments, the expression vector is a lentivirus.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87: 2264-2268, 1990, modified as in Proc. Natl. Acad. Sci. USA 90:5873-5877, 1993). Such a formula is incorporated into the basic local alignment search tool (BLAST) programs of Altschul et al. (J. Mol. Biol. 215: 403-410, 1990). Percent homology of sequences can be determined using the most recent version of BLAST, as of the filing date of this application.

The nucleic acids and vectors encoding the antibodies described herein can be used to infect, transfect, transform, or otherwise render a suitable cell transgenic for the nucleic acid, thus enabling the recombinant production of antibodies for commercial or therapeutic uses. Standard cell lines and methods for the production of antibodies from a large scale cell culture are known in the art. See e.g., Li et al., “Cell culture processes for monoclonal antibody production.” Mabs. 2010 September-October; 2(5): 466-477. In certain embodiments, the cell is a eukaryotic cell. In certain embodiments, the eukaryotic cell is a mammalian cell. In certain embodiments, the mammalian cell is a cell line useful for producing antibodies, such as a Chines Hamster Ovary cell (CHO) cell, an NSO murine myeloma cell, or a PER.C6® cell. In certain embodiments, the nucleic acid encoding the antibody is integrated into a genomic locus of a cell useful for producing antibodies. In certain embodiments, a method of making any of the IGF-1R antagonist antibodies described herein comprises culturing a cell comprising a nucleic acid encoding the antibody under conditions in vitro sufficient to allow production and secretion of said antibody.

The IGF-1R antagonist antibodies can be made from a master cell bank. In certain embodiments, a master cell bank comprises: (a) a mammalian cell line comprising a nucleic acid encoding an antibody described herein integrated at a genomic location; and (b) a cryoprotectant. In certain embodiments, the cryoprotectant comprises glycerol or DMSO. In certain embodiments, the master cell bank is contained in a suitable vial or container able to withstand freezing by liquid nitrogen.

Methods of making any of the IGF-1R antagonist antibodies described herein may comprise incubating a cell or cell-line comprising a nucleic acid encoding the antibody in a cell culture medium under conditions sufficient to allow for expression and secretion of the antibody, and further harvesting the antibody from the cell culture medium. The harvesting can further comprise one or more purification steps to remove live cells, cellular debris, non-antibody proteins or polypeptides, undesired salts, buffers, and medium components. In certain embodiments, the additional purification step(s) include centrifugation, ultracentrifugation, protein A, protein G, protein A/G, or protein L purification, and/or ion exchange chromatography.

“Treat,” “treatment,” or “treating,” as used herein refers to, e.g., a deliberate intervention to a physiological disease state resulting in the reduction in severity of a disease or condition; the reduction in the duration of a condition course; the amelioration or elimination of one or more symptoms associated with a disease or condition; or the provision of beneficial effects to a subject with a disease or condition. Treatment does not require curing the underlying disease or condition.

A “therapeutically effective amount,” “effective dose,” “effective amount,” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “affected with a disease or disorder,” “afflicted with a disease or disorder,” or “having a disease or disorder” are used interchangeably herein and refer to a subject or patient with any disease, disorder, syndrome or condition. No increased or decreased level of severity of the disorder is implied by the use of one of the terms as compared to the other.

As used herein, “pharmaceutically acceptable” with reference to a “carrier,” “excipient,” or “diluent” includes any and all solvents, dispersion media, coatings, antibacterial and antifungal agents, isotonic and absorption delaying agents, and the like that are physiologically compatible. In some aspects, the carrier is suitable for intravenous, intramuscular, subcutaneous, parenteral, spinal or epidermal administration (e.g., by injection or infusion). Depending on the route of administration, the active compound, i.e., antibody, can be coated in a material to protect the compound from the action of acids and other natural conditions that can inactivate the compound.

The pharmaceutical compounds described herein can include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66: 1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, “Thyroid Eye Disease” (TED), “Thyroid-associated Ophthalmopathy” (TAO), “Thyroid Inflammatory Eye Disease (TIED),” “Graves' Ophthalmopathy” (GO) or “Graves' Orbitopathy” (GO) refer to the same disorder or condition and are used interchangeably. They all refer to the inflammatory orbital pathology associated with some autoimmune thyroid disorders, most commonly with “Graves' Disease” (GD), but sometimes with other diseases, e.g. Hashimoto's thyroiditis.

As used herein, the terms “proptosis” and “exophthalmos” are used interchangeably and refer to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Proptosis or exophthalmos can be the result of several disease processes, including infections, inflammations, tumors, trauma, metastases, endocrine lesions, vascular diseases & extra orbital lesions. TED (TAO or GO) is currently recognized as the most common cause of proptosis in adults. Exophthalmos can be either bilateral, as is often seen in TED (TAO or GO), or unilateral (as is often seen in an orbital tumor).

Measurement of the degree of exophthalmos (i.e. proptosis) can be performed using an exophthalmometer, an instrument used for measuring the degree of forward displacement of the eye. The device allows measurement of the forward distance of the lateral orbital rim to the front of the cornea.

Computed tomography (CT) scanning and magnetic resonance imaging (MRI) may also be used in evaluating the degree of exophthalmos or proptosis. CT scanning is an excellent imaging modality for the diagnosis of TED (TAO or GO). In addition to allowing visualization of the enlarged extraocular muscles, CT scans provide the surgeon or clinician with depictions of the bony anatomy of the orbit when an orbital decompression is required. MRI, with its multi-planar and inherent contrast capabilities, provides excellent imaging of the orbital contents without the radiation exposure associated with CT scan studies. MRI provides better imaging of the optic nerve, orbital fat, and extraocular muscle, but CT scans provide better views of the bony architecture of the orbit.

Orbital ultrasonography can also be a used for the diagnosis and evaluation of TED (TAO or GO), because it can be performed quickly and with a high degree of confidence. High reflectivity and enlargement of the extraocular muscles are assessed easily, and serial ultrasonographic examinations can also be used to assess progression or stability of the ophthalmopathy.

Based on the technologies currently available, such as those described above, one of skill in the art would be capable of determining the best modality for diagnosing and evaluating the extent of proptosis or exophthalmos.

Although it is generally accepted that the normal range of proptosis is 12-21 mm, it must be noted that the value for a normal person varies by age, gender and race. For example, in normal adult white males, the average distance of globe protrusion is 16.5 mm, with the upper limit of normal at 21.7 mm. In adult African Americans it averages 18.2 mm, with an upper normal limit of 24.1 mm in males and 22.7 mm in females. In Mexican adults, males averaged 15.2 mm and females averaged 14.8 mm and in Iran, for the age group of 20-70 years, the average was 14.7 mm. In Taiwanese adults, comparing normal subjects to those with Graves' Ophthalmopathy, the normal group had an average reading of 13.9 mm versus 18.3 mm for the TAO group.

Even within a group of people, there can be variability. Four ethnic groups in Southern Thailand had exophthalmometry measurement averages ranging from 15.4 mm to 16.6 mm. In 2,477 Turkish patients, the median measurement was 13 mm, with an upper limit of 17 mm; and in a Dutch study, the upper limit was 20 mm in males and 16 mm in females.

Although the average and upper limits for exophthalmos or proptosis vary widely, it is accepted in the field that a difference greater than 2 mm between the eyes is significant and not normal.

One of skill in the art, for example an ophthalmologist, surgeon or other clinician skilled in the knowledge and treatment of eye disorders, would know what a normal value of proptosis is based on the age, gender and race of the subject and have the ability to diagnose or evaluate the presence or absence of proptosis as well as track its progression.

### Activity Measures or Assessments

Several classification systems have been conceived to assess the clinical manifestations of TED (TAO or GO). In 1969, Werner reported the NOSPECS Classification (No physical signs or symptoms, Only signs, Soft tissue involvement, Proptosis, Extraocular muscle signs, Corneal involvement, and Sight loss) (Werner, S. C. American Journal of Ophthalmology, 1969, 68, no. 4, 646-648.)

The modified NOSPECS was also published by Werner in 1977 and has been broadly used since then (Werner, S. C. American Journal of Ophthalmology, 1977, 83, no. 5, 725-727). This classification grades for clinical severity and does not provide a means of distinguishing active TAO (inflammatory progressive) from inactive TAO (noninflammatory stationary). Therefore, the indication for treatments used to be based exclusively in the severity of symptoms without consideration whether the disease was active or inactive. In 1989, Mourits et al. described the Clinical Activity Score (CAS) (Mourits et al., British Journal of Ophthalmology, 1989, 73, no. 8, 639-644) as a way of assessing the degree of active disease. This score, based on the classical signs of acute inflammation (pain, redness, swelling, and impaired function) was proposed as a clinical classification to discriminate easily between active and inactive disease and was modified in 1997 (Mourits et al., Clinical Endocrinology, 1997. 47, no. 1, 9-14). This protocol is further described below.

As used herein, the term CAS refers to the protocol described and scored as disclosed below. The CAS is a tool that typically consists of the following seven components:
1. spontaneous retrobulbar pain,
2. pain on attempted eye movements (upward, side-to-side, and downward gazes),
3. conjunctival redness,
4. redness of the eyelids,
5. chemosis (conjunctival swelling/edema),
6. swelling of the caruncle/plica, and
7. swelling of the eyelids.
   Each component is scored as present (1 point) or absent (0 points) for each eye or the most severely affected eye. The sum of all points defines clinical activity and provides the CAS. The score at each efficacy assessment is the sum of all items present; giving a range of 0-7, where 0 or 1 constitutes inactive disease and 7 severe active ophthalmopathy. A change of >2 points is considered clinically meaningful. A ten-item CAS is also sometimes used, which includes the following additional three components:
8. increase of >2 mm proptosis,
9. decrease in uniocular ocular excursion in any one direction of >8 degrees, and
10. decrease of acuity equivalent to 1 Snellen line.
   For patients assessed for the first time only items 1-7 are scored. A CAS≥3/7 indicates active GO or TED. For patients that are assessed for the second or subsequent time (typically, 1-3 months later), items 8-10 are also scored; and a CAS≥4/10 indicates active disease. In clinical trials, the 7-item scale is generally used, being more amenable to longitudinal studies involving multiple assessments.

Item 1, spontaneous orbital pain could be a painful, or oppressive feeling on, or behind, the globe. This pain may be caused by the rise in intraorbital pressure, when the orbital tissues volume increases through excess synthesis of extracellular matrix, fluid accumulation, and cellular infiltration and expansion. Item 2, gaze evoked orbital pain, could be pain in the eyes when looking, or attempting to look, up, down or sideways, i.e., pain with upward, downward, or lateral eye movement, or when attempting upward, downward, or lateral gaze. This kind of pain could arise from the stretching of the inflamed muscle(s), especially on attempted up-gaze. The ‘stretching pain’ cannot be provoked by digital pressing on the eyeball, as would be expected if it were a manifestation of the raised intraorbital pressure. Both kinds of pain can be reduced after anti-inflammatory treatment. These kinds of pain are therefore considered to be directly related to autoimmune inflammation in the orbit and thus useful in assessing TAO activity.

Swelling in TED (TAO or GO) is seen as chemosis (edema of the conjunctiva) and swelling of the caruncule and/or plica semilunaris. Both are signs of TED activity. Swollen eyelids can be caused by edema, fat prolapse through the orbital septum, or fibrotic degeneration. In addition to swelling, other symptoms indicative of active TED include redness and/or pain of the conjunctiva, eyelid, caruncule and/or plica semilunaris.

Other grading systems have also been developed for the assessment of TED (TAO or GO). The VISA Classification (vision, inflammation, strabismus, and appearance) (Dolman, P. J., and Rootman, J., Ophthalmic Plastic and Reconstructive Surgery, 2006, 22, no. 5, 319-324 and Dolman, P. J., Best Practice & Research Clinical Endocrinology & Metabolism, 2012, 26, no. 3, 229-248) and the European Group of Graves' Orbitopathy (EUGOGO) Classification (Bartalena, L., et al., European Journal of Endocrinology, 2008, 158, no. 3, 273-285) are two such examples. Both systems are grounded in the NO SPECS and CAS classifications and use indicators to assess the signs of activity and the degree of severity. More importantly, they allow the clinician to guide the treatment of the patient with GO. VISA is more commonly used in North America and Canada while EUGOGO is in Europe. Since the VISA and EUGOGO protocols are not interchangeable, only one of them should be employed as a reference in a specific patient.

### Graves Ophthalmopathy Quality of Life (GO-QoL)

In addition to proptosis (or exophthalmos) and CAS, quality of life (QoL) can also be evaluated in patients with TED with the use of the Graves' ophthalmopathy quality of life (GO-QoL) questionnaire. This questionnaire is designed to determine the improved quality of life after treatment. In some embodiments, the questionnaire may determine the decreased or lack of side effects after being treated with an antibody, or an antigen binding fragment thereof, according to the methods disclosed herein, as compared to treatment with glucocorticoids.

The GO-QoL questionnaire has two self-assessment subscales. The first relates to the impact of visual function on daily activities, while the second relates to the impact of self-perceived appearance. The visual function subscale has 8 questions which are answered with one of the three following choices: (i) Yes—seriously limited, (ii) yes—a little limited, or (iii) no—not at all limited. The appearance subscale also has 8 questions which are answered with one of the three following choices: (i) Yes—very much so; (ii) Yes—a little; or (iii) No—not at all. Each question is scored 0-2, respectively, and the total raw score is then mathematically transformed to a 0-100 scale, where 0 represents the most negative impact on quality of life, and 100 represents no impact. A change of ≥8 points on the 0-100 scale is considered to be clinically meaningful. The combined score takes raw scores from both subscales and again transforms them to a single 0-100 scale.

### Severity Measures

To assess the severity of TED in a patient, one or more of the following measures can be employed:
For lid aperture, the distance between the lid margins is measured (in mm) with the patient looking in the primary position, sitting relaxed, and with distant fixation.
For swelling of the eyelids, the measure/evaluation is either “absent/equivocal,” “moderate,” or “severe.”
Redness of the eyelids is either absent or present.
Redness of the conjunctivae is either absent or present.
Conjunctival edema is either absent or present.
Inflammation of the caruncle or plica is either absent or present.
Exophthalmos or proptosis can be measured in millimeter using the same Hertel exophthalmometer and same intercanthal distance for an individual patient.
Subjective diplopia is scored from 0 to 3 using the Gorman score according to the following scale:
   0=no diplopia;
   1=intermittent, i.e., diplopia in primary position of gaze, when tired or when first awakening;
   2=inconstant, i.e., diplopia at extremes of gaze;
   3=constant, i.e., continuous diplopia in primary or reading position.
For eye muscle involvement, the ductions are measured in degrees.
Corneal involvement is either absent/punctate or keratopathy/ulcer.
For optic nerve involvement, i.e., best-corrected visual acuity, color vision, optic disc, relative afferent pupillary defect, the condition is either absent or present. In addition, visual fields are checked if optic nerve compression was suspected.

### Severity Classification

Severity of thyroid eye disease is classified into three different categories according to various clinical guidelines (see, e.g., Burch et al., Eur Thyroid J., Vol. 11(6):e220189, 2022; and Bartalena et al., Eur J Endocrinol., Vol. 185(4):G43-G67, 2021): (i) mild thyroid eye disease, (ii) moderate to severe thyroid eye disease, and (iii) sight-threatening thyroid eye disease.

Sight-threatening thyroid eye disease: Patients with dysthyroid optic neuropathy (DON) and/or corneal breakdown, and/or globe subluxation. This category warrants immediate intervention.

Moderate-to-severe thyroid eye disease: Patients without sight-threatening disease whose thyroid eye disease has sufficient impact on daily life to justify the risks of medical or surgical intervention. Patients with moderate-to-severe thyroid eye disease usually have any one or more of the following: lid retraction ≥2 mm, moderate or severe soft tissue involvement, exophthalmos (proptosis) ≥3 mm above normal for race and gender, and inconstant or constant diplopia (Gorman score 2-3).

Mild thyroid eye disease: Patients whose features of thyroid eye disease have only a minor impact on daily life insufficient to justify immunosuppressive or surgical treatment. Patients with mild thyroid eye disease usually have only one or more of the following: minor lid retraction (<2 mm), mild soft tissue involvement, exophthalmos <3 mm above normal for race and gender, transient or no diplopia, and corneal exposure responsive to lubricants.

### Assessment of Gorman Grading of Diplopia

The Gorman assessment of subjective diplopia includes four categories: no diplopia (absent), diplopia when the patient is tired or awakening (intermittent), diplopia at extremes of gaze (inconstant), and continuous diplopia in the primary or reading position (constant). Patients are scored according to which grade of diplopia they are experiencing. An improvement of ≥1 grade is considered clinically meaningful.

Additional testing, including clinical trial protocols and criteria and the lead-in study, which can be performed to determine efficacy for the treatment of TED can be found in US20190225696A1, which is hereby incorporated by reference in its entirety.

Further, the IGR-1R antagonist antibodies described herein may be useful for the treatment of TED in subjects who were either proptosis non-responders (<2 mm reduction in proptosis in the study eye) after a course of treatment with a prior therapeutic agent for TED or were proptosis responders after a course of treatment with a prior therapeutic agent for TED but meet the criteria for re-treatment due to relapse (e.g. a new onset of diplopia, an increase in proptosis of 2 mm or greater in at least one eye, and/or a CAS of 4 or greater (on either the 7-component or 10-component scale) in at least one eye.

### IGF-1R Antagonist Antibodies

Described herein are methods of using an IGF-1R antagonistic antibody or antigen binding fragment thereof to treat TED or GO. IGF-1R antagonistic antibodies and pharmaceutical compositions comprising IGF-1R antagonistic antibodies can be used to treat TED in its active, inactive or chronic form.

As used herein, the term “IGF-1R antagonist antibody” refers to an antibody that specifically binds to human IGF-1R and inhibits its activation and downstream signaling by any of its ligands (e.g. IGF-1 and IGF-2). In certain embodiments, the antibody of the present disclosure inhibits activation of the IGF-1R. In certain embodiments, the inhibitor of IGF-1R biological function may exert its inhibitory function by binding to IGF-1R. In certain embodiments, the inhibitor of IGF-1R biological function may exert its inhibitory function by inhibiting IGF-1R signaling. In certain embodiments, the inhibitor of IGF-1R biological function may exert its inhibitory function by preventing IGF-1R autophosphorylation. In certain embodiments, the inhibitor of IGF-1R biological function may exert its inhibitory function by inhibiting IGF-1R signaling by inhibiting signaling downstream of IGF-1R. In certain embodiments, the inhibitor of IGF-1R biological function may exert its inhibitory function in the presence of ligand (e.g., insulin or insulin-like growth factor 1 or 2). In preferred embodiments, the antibodies, or antigen binding fragments thereof, used in the methods disclosed herein inhibit the binding of IGF-I and IGF-II to IGF-1R. The inhibition can be measured as IC₅₀ in an assay for binding of IGF-I/IGF-II to IGF-1R on cells. Such an assay is known to one of skill in the art and is described, for example, in U.S. Pat. No. 7,579,157, which is incorporated by reference herein in its entirety.

Described herein in one aspect is a method of treating TED or GO in an individual or patient in need thereof, the method comprising administering an antibody or antigen binding fragment thereof that binds insulin-like growth factor 1 receptor (IGF-1R), wherein the antibody or antigen binding fragment thereof comprises: an immunoglobulin heavy chain CDR1 (HCDR1) comprising the amino acid sequence of any one of SEQ ID NOs: 30 to 33; an immunoglobulin heavy chain CDR2 (HCDR2) comprising the amino acid sequence of any one of SEQ ID NOs: 34 to 40; an immunoglobulin heavy chain CDR3 (HCDR3) comprising the amino acid sequence of any one of SEQ ID NOs: 41 or 42; an immunoglobulin light chain CDR1 (LCDR1) comprising the amino acid sequence of any one of SEQ ID NOs: 43 to 45; an immunoglobulin light chain CDR2 (LCDR2) comprising the amino acid sequence of SEQ ID NO: 46; and/or an immunoglobulin light chain CDR3 (LCDR3) comprising the amino acid sequence of any one of SEQ ID NOs: 47 to 49; wherein the antibody or antigen binding fragment thereof does not comprise an immunoglobulin heavy chain variable region identical to SEQ ID NO: 1 and/or an immunoglobulin light chain variable region identical to SEQ ID NO: 2.

Described herein in one aspect is a method of treating TED or GO in an individual or patient in need thereof, the method comprising administering an antibody or antigen binding fragment thereof that binds IGF-1R, wherein the antibody or antigen binding fragment thereof comprises: (a) an immunoglobulin heavy chain CDR1 (HCDR1) comprising the amino acid sequence SX₁GMH (SEQ ID NO: 71), wherein X₁ is H, Y, A, or T; (b) an immunoglobulin heavy chain CDR2 (HCDR2) comprising the amino acid sequence X₁IX₂X₃DX₄SX₅TYYADSVRG (SEQ ID NO:72), wherein X₁ is I, T, or Y, X₂ is W, N, or A, X₃ is F, H, A, or G, X₄ is G or A, X₅ is S or T; (c) an immunoglobulin heavy chain CDR3 (HCDR3) comprising the amino acid sequence ELX₁RRYFDL (SEQ ID NO: 73), wherein X₁ is G or N; (d) an immunoglobulin light chain CDR1 (LCDR1) comprising the amino acid sequence RASQSVSSX₁LA (SEQ ID NO: 74), wherein X₁ is Y, A, or T; (e) an immunoglobulin light chain CDR2 (LCDR2) comprising the amino acid sequence DASKRAT (SEQ ID NO: 46); and/or an immunoglobulin light chain CDR3 (LCDR3) comprising the amino acid sequence QQRX₁KX₂PPWT (SEQ ID NO: 75), wherein X₁ is S or G, X₂ is Y or W; wherein the antibody or antigen binding fragment thereof does not comprise an immunoglobulin heavy chain variable region identical to SEQ ID NO: 1 and/or an immunoglobulin light chain variable region identical to SEQ ID NO: 2.

In certain embodiments, the IGF-1R antagonist antibody or antigen binding fragment thereof administered according to the methods of the invention comprises an immunoglobulin heavy chain variable region comprising an HCDR1, an HCDR2, and an HCDR3 and an immunoglobulin light chain variable region comprising an LCDR1, an LCDR2, and an LCDR3, wherein:
(a) HCDR1, HCDR2 and HCDR3 comprise the amino acid sequences of SEQ ID NOs: 30, 35, and 41, respectively, and LCDR1, LCDR2, and LCDR3 comprise the amino acid sequences of SEQ ID NOs: 43, 46, and 48, respectively;
(b) HCDR1, HCDR2 and HCDR3 comprise the amino acid sequences of SEQ ID NOs: 33, 38, and 41, respectively, and LCDR1, LCDR2, and LCDR3 comprise the amino acid sequences of SEQ ID NOs: 45, 46, and 48, respectively;
(c) HCDR1, HCDR2 and HCDR3 comprise the amino acid sequences of SEQ ID NOs: 30, 34, and 41, respectively, and LCDR1, LCDR2, and LCDR3 comprise the amino acid sequences of SEQ ID NOs: 45, 46, and 48, respectively;
(d) HCDR1, HCDR2 and HCDR3 comprise the amino acid sequences of SEQ ID NOs: 31, 35, and 42, respectively, and LCDR1, LCDR2, and LCDR3 comprise the amino acid sequences of SEQ ID NOs: 45, 46, and 48, respectively;
(e) HCDR1, HCDR2 and HCDR3 comprise the amino acid sequences of SEQ ID NOs: 33, 40, and 41, respectively, and LCDR1, LCDR2, and LCDR3 comprise the amino acid sequences of SEQ ID NOs: 45, 46, and 48, respectively;
(f) HCDR1, HCDR2 and HCDR3 comprise the amino acid sequences of SEQ ID NOs: 33, 37, and 41, respectively, and LCDR1, LCDR2, and LCDR3 comprise the amino acid sequences of SEQ ID NOs: 45, 46, and 48, respectively;
(g) HCDR1, HCDR2 and HCDR3 comprise the amino acid sequences of SEQ ID NOs: 32, 36, and 41, respectively, and LCDR1, LCDR2, and LCDR3 comprise the amino acid sequences of SEQ ID NOs: 45, 46, and 48, respectively;
(h) HCDR1, HCDR2 and HCDR3 comprise the amino acid sequences of SEQ ID NOs: 33, 35, and 41, respectively, and LCDR1, LCDR2, and LCDR3 comprise the amino acid sequences of SEQ ID NOs: 45, 46, and 48, respectively;
(i) HCDR1, HCDR2 and HCDR3 comprise the amino acid sequences of SEQ ID NOs: 30, 40, and 41, respectively, and LCDR1, LCDR2, and LCDR3 comprise the amino acid sequences of SEQ ID NOs: 44, 46, and 48, respectively; or
(j) HCDR1, HCDR2 and HCDR3 comprise the amino acid sequences of SEQ ID NOs: 33, 39, and 41, respectively, and LCDR1, LCDR2, and LCDR3 comprise the amino acid sequences of SEQ ID NOs: 43, 46, and 47, respectively.

In some embodiments, the IGF-1R antagonist antibody or antigen binding fragment thereof administered according to the methods of the invention comprises an immunoglobulin heavy chain variable region comprising an amino acid sequence that is at least 90% identical to an amino acid sequence selected from SEQ ID NOs: 3, 5, 7, 9, 11, 13, 15, 17, 19, and 21. In these and other embodiments, the IGF-1R antagonist antibody or antigen binding fragment thereof administered according to the methods of the invention comprises an immunoglobulin light chain variable region comprising an amino acid sequence that is at least 90% identical to an amino acid sequence selected from SEQ ID NOs: 4, 6, 8, 10, 12, 14, 16, 18, 20, and 22.

In certain embodiments of the methods of the invention, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 3; and wherein the immunoglobulin light chain variable region comprises an amino acid sequence that is at least about 90%, 95%, 97%, 99%, or 100% identical to that set forth in SEQ ID NO: 4. In certain other embodiments, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 5; and wherein the immunoglobulin light chain variable region comprises an amino acid sequence that is at least about 90%, 95%, 97%, 99%, or 100% identical to that set forth in SEQ ID NO: 6. In some embodiments, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 7; and wherein the immunoglobulin light chain variable region comprises an amino acid sequence that is at least about 90%, 95%, 97%, 99%, or 100% identical to that set forth in SEQ ID NO: 8. In other embodiments, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 9; and wherein the immunoglobulin light chain variable region comprises an amino acid sequence that is at least about 90%, 95%, 97%, 99%, or 100% identical to that set forth in SEQ ID NO: 10. In certain embodiments, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 11; and wherein the immunoglobulin light chain variable region comprises an amino acid sequence that is at least about 90%, 95%, 97%, 99%, or 100% identical to that set forth in SEQ ID NO: 12. In certain other embodiments, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 13; and wherein the immunoglobulin light chain variable region comprises an amino acid sequence that is at least about 90%, 95%, 97%, 99%, or 100% identical to that set forth in SEQ ID NO: 14. In some embodiments, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 15; and wherein the immunoglobulin light chain variable region comprises an amino acid sequence that is at least about 90%, 95%, 97%, 99%, or 100% identical to that set forth in SEQ ID NO: 16. In other embodiments, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 17; and wherein the immunoglobulin light chain variable region comprises an amino acid sequence that is at least about 90%, 95%, 97%, 99%, or 100% identical to that set forth in SEQ ID NO: 18. In certain embodiments, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 19; and wherein the immunoglobulin light chain variable region comprises an amino acid sequence that is at least about 90%, 95%, 97%, 99%, or 100% identical to that set forth in SEQ ID NO: 20. In certain other embodiments, the IGF-1R antagonist antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 21; and wherein the immunoglobulin light chain variable region comprises an amino acid sequence that is at least about 90%, 95%, 97%, 99%, or 100% identical to that set forth in SEQ ID NO: 22.

In some embodiments, the IGF-1R antagonist antibody or antigen binding fragment thereof administered according to the methods of the invention comprise an immunoglobulin heavy chain variable region and an immunoglobulin light chain variable region, wherein:
(a) the immunoglobulin heavy chain variable region comprises the amino acid sequence of SEQ ID NO: 3 and the immunoglobulin light chain variable region comprises the amino acid sequence of SEQ ID NO: 4;
(b) the immunoglobulin heavy chain variable region comprises the amino acid sequence of SEQ ID NO: 5 and the immunoglobulin light chain variable region comprises the amino acid sequence of SEQ ID NO: 6;
(c) the immunoglobulin heavy chain variable region comprises the amino acid sequence of SEQ ID NO: 7 and the immunoglobulin light chain variable region comprises the amino acid sequence of SEQ ID NO: 8;
(d) the immunoglobulin heavy chain variable region comprises the amino acid sequence of SEQ ID NO: 9 and the immunoglobulin light chain variable region comprises the amino acid sequence of SEQ ID NO: 10;
(e) the immunoglobulin heavy chain variable region comprises the amino acid sequence of SEQ ID NO: 11 and the immunoglobulin light chain variable region comprises the amino acid sequence of SEQ ID NO: 12;
(f) the immunoglobulin heavy chain variable region comprises the amino acid sequence of SEQ ID NO: 13 and the immunoglobulin light chain variable region comprises the amino acid sequence of SEQ ID NO: 14;
(g) the immunoglobulin heavy chain variable region comprises the amino acid sequence of SEQ ID NO: 15 and the immunoglobulin light chain variable region comprises the amino acid sequence of SEQ ID NO: 16;
(h) the immunoglobulin heavy chain variable region comprises the amino acid sequence of SEQ ID NO: 17 and the immunoglobulin light chain variable region comprises the amino acid sequence of SEQ ID NO: 18;
(i) the immunoglobulin heavy chain variable region comprises the amino acid sequence of SEQ ID NO: 19 and the immunoglobulin light chain variable region comprises the amino acid sequence of SEQ ID NO: 20; or
(j) the immunoglobulin heavy chain variable region comprises the amino acid sequence of SEQ ID NO: 21 and the immunoglobulin light chain variable region comprises the amino acid sequence of SEQ ID NO: 22.

In some embodiments, the IGF-1R antagonist antibody or antigen binding fragment thereof used in the methods of the invention is an IgG antibody or antigen binding fragment. For instance, in some such embodiments, the IGF-1R antagonist antibody comprises a constant region from a human IgG immunoglobulin, such as a human IgG1, IgG2, IgG3, or IgG4 immunoglobulin. In one embodiment, the IGF-1R antagonist antibody comprises a constant region from a human IgG1 immunoglobulin. In these and other embodiments, the IGF-1R antagonist antibody comprises a M252Y, a S254T, and a T256E substitution according to EU numbering in one or both heavy chain constant regions.

In certain embodiments, the IGF-1R antagonist antibody or antigen binding fragment thereof administered according to the methods of the invention is a monoclonal antibody or antigen binding fragment thereof. In some embodiments, the antibody or antigen binding fragment thereof is chimeric or humanized. In other embodiments, the antibody or antigen binding fragment thereof is a human antibody or antigen binding fragment thereof. In certain embodiments, the IGF-1R antagonist antibody used in the methods of the invention is a full-length antibody.

In certain embodiments, the antigen binding fragment is a Fab, F(ab)₂, or a single chain variable fragment (scFv). In certain other embodiments, the antibody or antigen binding fragment inhibits signaling through IGF-1R. In some embodiments, the antibody or antigen binding fragment thereof binds to IGF-1R with a K_{D} of less than 5×10⁻⁹ M. In other embodiments, the antibody binds to IGF-1R with a K_{D} of less than 1×10⁻⁹ M. In certain other embodiments, the antibody binds to IGF-1R with a K_{D} of less than 5×10⁻¹⁰ M. In particular embodiments, the K_{D} of the antibody or antigen binding fragment for IGF-1R is determined by biolayer interferometry, such as the method described in Example 1. In certain embodiments, the antibody possesses a half-life of 14 days or longer in a human. In certain other embodiments, the antibody possesses a half-life of 21 days or longer in a human.

In some embodiments, the IGF-1R antagonist antibody administered according to the methods of the invention is a full-length antibody and has the structure of a native tetrameric immunoglobulin—that is the IGF-1R antagonist antibody comprises two immunoglobulin heavy chains and two immunoglobulin light chains. In some such embodiments, each immunoglobulin heavy chain comprises an amino acid sequence that is at least 90% identical to an amino acid sequence selected from SEQ ID NOs: 51, 53, 55, 57, 59, 61, 63, 65, 67, and 69 and each immunoglobulin light chain comprises an amino acid sequence that is at least 90% identical to an amino acid sequence selected from SEQ ID NOs: 52, 54, 56, 58, 60, 62, 64, 66, 68, and 70. In other such embodiments, the IGF-1R antagonist antibody comprises two immunoglobulin heavy chains and two immunoglobulin light chains, wherein
(a) each immunoglobulin heavy chain comprises the amino acid sequence of SEQ ID NO: 51 and each immunoglobulin light chain comprises the amino acid sequence of SEQ ID NO: 52;
(b) each immunoglobulin heavy chain comprises the amino acid sequence of SEQ ID NO: 53 and each immunoglobulin light chain comprises the amino acid sequence of SEQ ID NO: 54;
(c) each immunoglobulin heavy chain comprises the amino acid sequence of SEQ ID NO: 55 and each immunoglobulin light chain comprises the amino acid sequence of SEQ ID NO: 56;
(d) each immunoglobulin heavy chain comprises the amino acid sequence of SEQ ID NO: 57 and each immunoglobulin light chain comprises the amino acid sequence of SEQ ID NO: 58;
(e) each immunoglobulin heavy chain comprises the amino acid sequence of SEQ ID NO: 59 and each immunoglobulin light chain comprises the amino acid sequence of SEQ ID NO: 60;
(f) each immunoglobulin heavy chain comprises the amino acid sequence of SEQ ID NO: 61 and each immunoglobulin light chain comprises the amino acid sequence of SEQ ID NO: 62;
(g) each immunoglobulin heavy chain comprises the amino acid sequence of SEQ ID NO: 63 and each immunoglobulin light chain comprises the amino acid sequence of SEQ ID NO: 64;
(h) each immunoglobulin heavy chain comprises the amino acid sequence of SEQ ID NO: 65 and each immunoglobulin light chain comprises the amino acid sequence of SEQ ID NO: 66;
(i) each immunoglobulin heavy chain comprises the amino acid sequence of SEQ ID NO: 67 and each immunoglobulin light chain comprises the amino acid sequence of SEQ ID NO: 68; or
(j) each immunoglobulin heavy chain comprises the amino acid sequence of SEQ ID NO: 69 and each immunoglobulin light chain comprises the amino acid sequence of SEQ ID NO: 70.

In certain embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 51; and wherein the immunoglobulin light chain comprises an amino acid sequence that is at least about 90%, 95%, 97%, 99%, or 100% identical to that set forth in SEQ ID NO: 52.

In certain other embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 53; and wherein the immunoglobulin light chain comprises an amino acid sequence that is at least about 90%, 95%, 97%, 99%, or 100% identical to that set forth in SEQ ID NO: 54.

In some embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 55; and wherein the immunoglobulin light chain comprises an amino acid sequence that is at least about 90%, 95%, 97%, 99%, or 100% identical to that set forth in SEQ ID NO: 56.

In other embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 57; and wherein the immunoglobulin light chain comprises an amino acid sequence that is at least about 90%, 95%, 97%, 99%, or 100% identical to that set forth in SEQ ID NO: 58.

In certain embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 59; and wherein the immunoglobulin light chain comprises an amino acid sequence that is at least about 90%, 95%, 97%, 99%, or 100% identical to that set forth in SEQ ID NO: 60.

In certain other embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 61; and wherein the immunoglobulin light chain comprises an amino acid sequence that is at least about 90%, 95%, 97%, 99%, or 100% identical to that set forth in SEQ ID NO: 62.

In some embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 63; and wherein the immunoglobulin light chain comprises an amino acid sequence that is at least about 90%, 95%, 97%, 99%, or 100% identical to that set forth in SEQ ID NO: 64.

In other embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 65; and wherein the immunoglobulin light chain comprises an amino acid sequence that is at least about 90%, 95%, 97%, 99%, or 100% identical to that set forth in SEQ ID NO: 66.

In certain embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 67; and wherein the immunoglobulin light chain comprises an amino acid sequence that is at least about 90%, 95%, 97%, 99%, or 100% identical to that set forth in SEQ ID NO: 68.

In certain other embodiments, the IGF-1R antagonist antibody comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 69; and wherein the immunoglobulin light chain comprises an amino acid sequence that is at least about 90%, 95%, 97%, 99%, or 100% identical to that set forth in SEQ ID NO: 70.

Described herein in one aspect is a method of treating TED or GO in an individual or patient in need thereof, the method comprising administering an antibody or antigen binding fragment thereof that binds IGF-1R, wherein the antibody or antigen binding fragment thereof comprises: (a) an HCDR1 comprising the amino acid sequence SHGMH (SEQ ID NO: 30); (b) an HCDR2 comprising the amino acid sequence YIWFDGSSTYYADSVRG (SEQ ID NO: 35); (c) an HCDR3 comprising the amino acid sequence ELGRRYFDL (SEQ ID NO: 41); (d) an LCDR1 comprising the amino acid sequence RASQSVSSALA (SEQ ID NO: 43); (e) an LCDR2 comprising the amino acid sequence DASKRAT (SEQ ID NO: 46); and/or (f) an LCDR3 comprising the amino acid sequence QQRSKYPPWT (SEQ ID NO: 48). In certain embodiments, the antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 17; and wherein the immunoglobulin light chain variable region comprises an amino acid sequence that is at least about 90%, 95%, 97%, 99%, or 100% identical to that set forth in SEQ ID NO: 18. In certain embodiments, the antibody or antigen binding fragment thereof comprises an immunoglobulin heavy chain variable region and an immunoglobulin light chain variable region, wherein the immunoglobulin heavy chain variable region comprises an amino acid sequence identical to that set forth in SEQ ID NO: 17; and wherein the immunoglobulin light chain variable region comprises an amino acid sequence identical to that set forth in SEQ ID NO: 18. In certain other embodiments, the antibody or antigen binding fragment thereof comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 51; and wherein the immunoglobulin light chain comprises an amino acid sequence that is at least about 90%, 95%, 97%, 99%, or 100% identical to that set forth in SEQ ID NO: 52. In certain embodiments, the antibody or antigen binding fragment thereof is an IgG antibody or antigen binding fragment thereof. In certain embodiments, the antigen binding fragment thereof is a Fab, F(ab)₂, or a single chain variable fragment (scFv). In certain other embodiments, the antibody is a full-length antibody. In certain embodiments, the antibody or antigen binding fragment thereof is chimeric or humanized. In certain other embodiments, the antibody or antigen binding fragment thereof is a human antibody or antigen binding fragment thereof. In some embodiments, the antibody or antigen binding fragment inhibits signaling through IGF-1R. In certain embodiments, the antibody or antigen binding fragment binds to IGF-1R with a K_{D} of less than 5×10⁻⁹ M. In other embodiments, the antibody or antigen binding fragment binds to IGF-1R with a K_{D} of less than 1×10⁻⁹ M. In certain embodiments, the antibody or antigen binding fragment binds to IGF-1R with a K_{D} of less than 5×10⁻¹⁰ M. In some embodiments, the antibody possesses a half-life of 14 days or longer in a human. In other embodiments, the antibody possesses a half-life of 21 days or longer in a human. In certain embodiments, the antibody comprises a M252Y, a S254T and a T256E substitution according to EU numbering in one or both heavy chain constant regions.

Described herein in one aspect is method of treating TED or GO in an individual or patient in need thereof, the method comprising administering an antibody or antigen binding fragment thereof that binds IGF-1R, wherein the antibody or antigen binding fragment thereof comprises: (a) an HCDR1 comprising the amino acid sequence SYGMH (SEQ ID N: 33); (b) an HCDR2 comprising the amino acid sequence IIWFDGSSTYYADSVRG (SEQ ID NO: 38); (c) an HCDR3 comprising the amino acid sequence ELGRRYFDL (SEQ ID NO: 41); (d) an LCDR1 comprising the amino acid sequence RASQSVSSYLA (SEQ ID NO: 45); (e) an LCDR2 comprising the amino acid sequence DASKRAT (SEQ ID NO: 46); and/or (f) an LCDR3 comprising the amino acid sequence QQRSKYPPWT (SEQ ID NO: 48). In certain embodiments, the antibody or antigen binding fragment thereof is an IgG antibody or antigen binding fragment thereof. In certain embodiments, the antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 7; and wherein the immunoglobulin light chain variable region comprises an amino acid sequence that is at least about 90%, 95%, 97%, 99%, or 100% identical to that set forth in SEQ ID NO: 8. In certain embodiments, the antibody or antigen binding fragment thereof comprises an immunoglobulin heavy chain variable region and an immunoglobulin light chain variable region, wherein the immunoglobulin heavy chain variable region comprises an amino acid sequence identical to that set forth in SEQ ID NO: 7; and wherein the immunoglobulin light chain variable region comprises an amino acid sequence identical to that set forth in SEQ ID NO: 8. In certain other embodiments, the antibody or antigen binding fragment thereof comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 57; and wherein the immunoglobulin light chain comprises an amino acid sequence that is at least about 90%, 95%, 97%, 99%, or 100% identical to that set forth in SEQ ID NO: 58. In certain embodiments, the antigen binding fragment thereof is a Fab, F(ab)₂, or a single chain variable fragment (scFv). In certain other embodiments, the antibody is a full-length antibody. In certain embodiments, the antibody or antigen binding fragment thereof is chimeric or humanized. In certain other embodiments, the antibody or antigen binding fragment thereof is a human antibody or antigen binding fragment thereof. In some embodiments, the antibody or antigen binding fragment inhibits signaling through IGF-1R. In certain embodiments, the antibody or antigen binding fragment binds to IGF-1R with a K_{D} of less than 5×10⁻⁹ M. In other embodiments, the antibody or antigen binding fragment binds to IGF-1R with a K_{D} of less than 1×10⁻⁹ M. In some embodiments, the antibody or antigen binding fragment binds to IGF-1R with a K_{D} of less than 5×10⁻¹⁰M. In certain embodiments, the antibody possesses a half-life of 14 days or longer in a human. In certain other embodiments, the antibody possesses a half-life of 21 days or longer in a human. In certain embodiments, the antibody comprises a M252Y, a S254T and a T256E substitution according to EU numbering in one or both heavy chain constant regions.

Described herein in one aspect is method of treating TED or GO in an individual or patient in need thereof, the method comprising administering an antibody or antigen binding fragment thereof that binds IGF-1R, wherein the antibody or antigen binding fragment thereof comprises: (a) an HCDR1 comprising the amino acid sequence SHGMH (SEQ ID NO: 30); (b) an HCDR2 comprising the amino acid sequence IIAGDASTTYYADSVRG (SEQ ID NO: 34); (c) an HCDR3 comprising the amino acid sequence ELGRRYFDL (SEQ ID NO: 41); (d) an LCDR1 comprising the amino acid sequence RASQSVSSYLA (SEQ ID NO: 45); (e) an LCDR2 comprising the amino acid sequence DASKRAT (SEQ ID NO: 46); and/or (f) an LCDR3 comprising the amino acid sequence QQRSKYPPWT (SEQ ID NO: 48). In certain embodiments, the antibody or antigen binding fragment thereof comprises an immunoglobulin heavy chain variable region and an immunoglobulin light chain variable region, wherein the immunoglobulin heavy chain variable region comprises an amino acid sequence that is at least about 90%, 95%, 97%, 99%, or 100% identical to that set forth in SEQ ID NO: 5; and wherein the immunoglobulin light chain variable region comprises an amino acid sequence that is at least about 90%, 95%, 97%, 99%, or 100% identical to that set forth in SEQ ID NO: 6. In certain embodiments, the antibody or antigen binding fragment thereof comprises an immunoglobulin heavy chain variable region and an immunoglobulin light chain variable region, wherein the immunoglobulin heavy chain variable region comprises an amino acid sequence identical to that set forth in SEQ ID NO: 5; and wherein the immunoglobulin light chain variable region comprises an amino acid sequence identical to that set forth in SEQ ID NO: 6. In certain other embodiments, the antibody or antigen binding fragment thereof comprises an immunoglobulin heavy chain and an immunoglobulin light chain, wherein the immunoglobulin heavy chain comprises an amino acid sequence that is at least about 90%, 95%, 97%, 99%, or 100% identical to that set forth in SEQ ID NO: 55; and wherein the immunoglobulin light chain comprises an amino acid sequence that is at least about 90%, 95%, 97%, 99%, or 100% identical to that set forth in SEQ ID NO: 56. In certain embodiments, the antibody or antigen binding fragment thereof is an IgG antibody or antigen binding fragment thereof. In certain embodiments, the antigen binding fragment thereof is a Fab, F(ab)₂, or a single chain variable fragment (scFv). In certain other embodiments, the antibody is a full-length antibody. In certain embodiments, the antibody or antigen binding fragment thereof is chimeric or humanized. In certain other embodiments, the antibody or antigen binding fragment thereof is a human antibody or antigen binding fragment thereof. In certain embodiments, the antibody or antigen binding fragment inhibits signaling through IGF-1R. In certain embodiments, the antibody or antigen binding fragment binds to IGF-1R with a K_{D} of less than 5×10⁻⁹ M. In certain other embodiments, the antibody or antigen binding fragment binds to IGF-1R with a K_{D} of less than 1×10⁻⁹ M. In some embodiments, the antibody or antigen binding fragment binds to IGF-1R with a K_{D} of less than 5×10⁻¹⁰ M. In certain embodiments, the antibody possesses a half-life of 14 days or longer in a human. In certain other embodiments, the antibody possesses a half-life of 21 days or longer in a human. In some embodiments, the antibody possesses a half-life of 25 days or longer in a human. In other embodiments, the antibody possesses a half-life of 30 days or longer in a human. In certain embodiments, the antibody comprises a M252Y, a S254T and a T256E substitution according to EU numbering in one or both heavy chain constant regions.

In one aspect, described herein, is a teprotumumab derivative with increased affinity, wherein the teprotumumab derivative comprises a substitution of the tryptophan at position 94 of SEQ ID NO: 2 to tyrosine.

IGF-1R signaling is perturbed (e.g., increased) in thyroid eye disease or Grave's ophthalmopathy and thus the IGF-1R antagonist antibodies described herein are potentially useful for the treatment of such diseases associated with this aberrant signaling. The IGF-1R antagonist antibodies described herein can be used to effectively treat individuals with IGF-1R disorders by inhibiting IGF-1R signaling. IGF-1R signaling occurs primarily through the PI3K and the RAS pathways. IGF-1R singling or inhibition of IGF-1R signaling can be determined by phosphorylation of IGF-1R. In certain embodiments, the antibodies described herein exhibit inhibition with an EC50 of 10 ng/mL or less. In certain embodiments, the antibodies described herein exhibit inhibition with an EC50 of 9 ng/mL or less. In certain embodiments, the antibodies described herein exhibit inhibition with an EC50 of 8 ng/mL or less. In certain embodiments, the antibodies described herein exhibit inhibition with an EC50 of 7 ng/mL or less. In certain embodiments, the antibodies described herein exhibit inhibition with an EC50 of 6 ng/mL or less. In certain embodiments, the antibodies described herein exhibit inhibition with an EC50 of 5 ng/mL or less. Such assays to determine EC50 are described herein and can be carried out with 200 ng/mL of recombinant human IGF-1 using 4×10⁴ NCI-H322 cells/well in flat bottom 96-well plates.

In certain embodiments of the methods of the invention, the IGF-1R antagonist antibodies can be administered to an individual or patient in need thereof (e.g., afflicted with an IGF-1R signaling disorder or disease associated with aberrant IGF-1R signaling, such as thyroid eye disease) by any route suitable for the administration of antibody-containing pharmaceutical compositions, such as, for example, by subcutaneous, intraperitoneal, intravenous, intramuscular, or intratumoral, etc. routes of administration. In certain embodiments of the methods of the invention, the IGF-1R antagonist antibodies are administered intravenously. In certain other embodiments, the IGF-1R antagonist antibodies are administered subcutaneously, e.g., by subcutaneous injection. In other embodiments, the IGF-1R antagonist antibodies are administered intratumorally.

In certain embodiments, the IGF-1R antagonist antibodies of this disclosure are administered at a therapeutically effective dosage and/or on a therapeutically acceptable schedule. In some embodiments, a therapeutically effective schedule is once weekly, once every two weeks, once every three weeks, once every four weeks, once every five weeks, once every six weeks, once every seven weeks, once every eight weeks, once every nine weeks, once every ten weeks, once every eleven weeks, or once every twelve weeks. In certain embodiments, the IGF-1R antagonist antibodies of this disclosure are administered from about 0.1 mg/kg per dose to about 50 mg/kg per dose. In some embodiments, the IGF-1R antagonist antibodies of this disclosure are administered from about 1 mg/kg per dose to about 50 mg/kg per dose. In other embodiments, the IGF-1R antagonist antibodies of this disclosure are administered from about 5 mg/kg per dose to about 50 mg/kg per dose. In certain embodiments, the IGF-1R antagonist antibodies of this disclosure are administered from about 5 mg/kg per dose to about 40 mg/kg per dose. In certain other embodiments, the IGF-1R antagonist antibodies of this disclosure are administered from about 5 mg/kg per dose to about 30 mg/kg per dose. In some embodiments, the IGF-1R antagonist antibodies of this disclosure are administered from about 5 mg/kg per dose to about 25 mg/kg per dose. In other embodiments, the IGF-1R antagonist antibodies of this disclosure are administered from about 5 mg/kg per dose to about 20 mg/kg per dose. In certain embodiments, the IGF-1R antagonist antibodies of this disclosure are administered from about 5 mg/kg per dose to about 15 mg/kg per dose. In certain other embodiments, the IGF-1R antagonist antibodies of this disclosure are administered from about 5 mg/kg per dose to about 10 mg/kg per dose. In certain embodiments, the IGF-1R antagonist antibodies of this disclosure are administered from about 10 mg/kg per dose to about 30 mg/kg per dose. In certain other embodiments, the IGF-1R antagonist antibodies of this disclosure are administered from about 10 mg/kg per dose to about 25 mg/kg per dose. In some embodiments, the IGF-1R antagonist antibodies of this disclosure are administered from about 10 mg/kg per dose to about 20 mg/kg per dose. In other embodiments, the IGF-1R antagonist antibodies of this disclosure are administered at about 0.1 mg/kg per dose. In certain embodiments, the IGF-1R antagonist antibodies of this disclosure are administered at about 1 mg/kg per dose. In certain other embodiments, the IGF-1R antagonist antibodies of this disclosure are administered at about 2 mg/kg per dose. In some embodiments, the IGF-1R antagonist antibodies of this disclosure are administered at about 3 mg/kg per dose. In other embodiments, the IGF-1R antagonist antibodies of this disclosure are administered at about 4 mg/kg per dose. In certain embodiments, the IGF-1R antagonist antibodies of this disclosure are administered at about 5 mg/kg per dose. In certain other embodiments, the IGF-1R antagonist antibodies of this disclosure are administered at about 10 mg/kg per dose. In some embodiments, the IGF-1R antagonist antibodies of this disclosure are administered at about 15 mg/kg per dose. In other embodiments, the IGF-1R antagonist antibodies of this disclosure are administered at about 20 mg/kg per dose. In certain embodiments, the IGF-1R antagonist antibodies of this disclosure are administered at about 25 mg/kg per dose. In certain other embodiments, the IGF-1R antagonist antibodies of this disclosure are administered at about 30 mg/kg per dose. In some embodiments, the IGF-1R antagonist antibodies of this disclosure are administered at about 35 mg/kg per dose. In certain embodiments, the IGF-1R antagonist antibodies of this disclosure are administered at about 40 mg/kg per dose. In some embodiments, the IGF-1R antagonist antibodies of this disclosure are administered at about 45 mg/kg per dose. In other embodiments, the IGF-1R antagonist antibodies of this disclosure are administered at about 50 mg/kg per dose.

In certain embodiments, the methods described herein comprise administering a first dose that is different than a subsequently administered dose. In certain embodiments, the first dose is a loading dose that is higher than a subsequent maintenance dose. In certain embodiments, the first dose is a dose that is lower than a subsequent dose.

In certain embodiments, a first dose is administered from about 0.1 mg/kg to about 30 mg/kg; and a subsequent dose is administered in a higher amount from about 0.1 mg/kg to about 30 mg/kg. In certain embodiments, a first dose is administered from about 1 mg/kg to about 30 mg/kg; and a subsequent dose is administered in a higher amount from about 1 mg/kg to about 30 mg/kg. In certain embodiments, a first dose is administered from about 5 mg/kg to about 30 mg/kg; and a subsequent dose is administered in a higher amount from about 5 mg/kg to about 30 mg/kg. In certain embodiments, a first dose is administered from about 5 mg/kg to about 25 mg/kg; and a subsequent dose is administered in a higher amount from about 5 mg/kg to about 25 mg/kg. In certain embodiments, a first dose is administered from about 5 mg/kg to about 10 mg/kg; and a subsequent dose is administered in a higher amount from about 5 mg/kg to about 10 mg/kg. In certain embodiments, a first dose is administered from about 10 mg/kg to about 20 mg/kg; and a subsequent dose is administered in a higher amount from about 10 mg/kg to about 20 mg/kg. In certain embodiments, a first dose is administered from about 15 mg/kg to about 25 mg/kg; and a subsequent dose is administered in a higher amount from about 15 mg/kg to about 25 mg/kg. In certain embodiments, a first dose is administered at about 10 mg/kg; and a subsequent dose is administered at about 20 mg/kg.

In certain embodiments, a dose is administered every 3 weeks for 8 total over 6 months. In certain embodiments, a first dose is administered from about 0.1 mg/kg to about 30 mg/kg; and for remaining 7 doses a subsequent dose is administered in a higher amount from about 0.1 mg/kg to about 30 mg/kg every 3 weeks over 6 months. In certain embodiments, a first dose is administered from about 1 mg/kg to about 30 mg/kg; and for remaining 7 doses a subsequent dose is administered in a higher amount from about 1 mg/kg to about 30 mg/kg every 3 weeks over 6 months. In certain embodiments, a first dose is administered from about 5 mg/kg to about 30 mg/kg; and for remaining 7 doses a subsequent dose is administered in a higher amount from about 5 mg/kg to about 30 mg/kg every 3 weeks over 6 months. In certain embodiments, a first dose is administered from about 5 mg/kg to about 25 mg/kg; and for remaining 7 doses a subsequent dose is administered in a higher amount from about 5 mg/kg to about 25 mg/kg every 3 weeks over 6 months. In certain embodiments, a first dose is administered from about 10 mg/kg to about 20 mg/kg; and for remaining 7 doses a subsequent dose is administered in a higher amount from about 10 mg/kg to about 20 mg/kg every 3 weeks over 6 months. In certain embodiments, a first dose is administered from about 15 mg/kg to about 25 mg/kg; and for remaining 7 doses a subsequent dose is administered in a higher amount from about 15 mg/kg to about 25 mg/kg every 3 weeks over 6 months. In certain embodiments, a first dose is administered at about 10 mg/kg; and for remaining 7 doses a subsequent dose is administered in a higher amount at about 20 mg/kg every 3 weeks over 6 months.

### Treatment of Thyroid Eve Disease

In certain embodiments, a patient to be treated according to the methods of the invention has or is diagnosed with active thyroid eye disease. Thus, the present invention includes methods of treating active thyroid eye disease in a patient in need thereof. Active thyroid eye disease generally refers to phases of the disease characterized by inflammation and tissue damage. In some embodiments, active thyroid eye disease can be diagnosed using the CAS as described above. A CAS can differentiate between active and inactive TED. A CAS of ≥3/7 on the 7-component scale and a CAS of ≥4/10 on the 10-component scale is indicative of active TED. Active TED may also be diagnosed if the patient has a history or documentation of progression of TED based on subjective or objective worsening of vision, soft tissue inflammation, motility, or proptosis independently of the CAS. In some embodiments, the patient with active TED to be treated according to the methods of the invention has a CAS of ≥3 on the 7-component scale (i.e. a CAS of 3 to 7) in at least one eye. In other embodiments, the patient to be treated with active TED according to the methods of the invention has a CAS of ≥4 on the 10-component scale (i.e. a CAS of 4 to 10) in at least one eye.

In certain embodiments, described herein is a method of treating an individual or patient with active thyroid eye disease, the method comprising administering to an individual with active thyroid eye disease (TED) an IGF-1R antagonist antibody as described herein, thereby treating the active TED. In certain embodiments, the individual or patient with active TED has a CAS of 3 or more. In certain embodiments, the individual or patient with active TED has a CAS of 3. In certain other embodiments, the individual or patient with active TED has a CAS of 4 or more. In certain embodiments, the individual or patient with active TED has a CAS of 5 or more. In certain other embodiments, the individual or patient with active TED has a CAS of 6 or more. In some embodiments, the individual or patient with active TED has a CAS of 7 or more. In certain embodiments, the individual or patient with active TED has a CAS of 8 or more. In certain other embodiments, the individual or patient with active TED has a CAS of 9 or more. In certain embodiments, the individual or patient with active TED has a CAS of 10. In some embodiments, the TED has been active for at least 1, 2, 3, 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 48, 50 or 60 months.

In certain embodiments, the methods described herein result in a reduced CAS of an individual or patient with active TED. In certain embodiments, administration of the IGF-1R antagonist antibody as described herein reduces an individual's or patient's CAS by 1 or more. In other embodiments, administration of the IGF-1R antagonist antibody as described herein reduces an individual's or patient's CAS by 2 or more. In some embodiments, administration of the IGF-1R antagonist antibody as described herein reduces an individual's or patient's CAS by 3 or more. In other embodiments, administration of the IGF-1R antagonist antibody as described herein reduces an individual's or patient's CAS by 4 or more. In certain embodiments, administration of the IGF-1R antagonist antibody as described herein reduces an individual's or patient's CAS by 5 or more. In certain other embodiments, administration of the IGF-1R antagonist antibody as described herein reduces an individual's or patient's CAS by 6 or more. In some embodiments, administration of the IGF-1R antagonist antibody as described herein reduces an individual's or patient's CAS by 7 or more. In other embodiments, administration of the IGF-1R antagonist antibody as described herein reduces an individual's or patient's CAS by 8 or more. In certain embodiments, administration of the IGF-1R antagonist antibody as described herein reduces an individual's or patient's CAS by 9 or more. In certain other embodiments, administration of the IGF-1R antagonist antibody as described herein reduces an individual's or patient's CAS by 10. In some embodiments, administration of the IGF-1R antagonist antibody as described herein reduces an individual's or patient's CAS to lower than 3. In certain embodiments, administration of the IGF-1R antagonist antibody as described herein reduces an individual's or patient's CAS to lower than 2. In certain other embodiments, administration of the IGF-1R antagonist antibody as described herein reduces an individual's or patient's CAS to 1 or 0. In some embodiments, administration of the IGF-1R antagonist antibody as described herein reduces an individual's or patient's CAS to 0. In other embodiments, administration of the IGF-1R antagonist antibody as described herein reduces an individual's or patient's CAS to 2. In certain embodiments, administration of the IGF-1R antagonist antibody as described herein reduces an individual's or patient's CAS to 1.

The CAS of an individual may be different in the different eyes of the individual. In some embodiments, the CAS is determined for the most severely affected eye. The methods used to treat TED may in certain instances result in reductions of the CAS in one or both eyes. The methods used to treat active TED may in certain instances be used in patients or individuals if both eyes have a CAS of greater than 2 or 3, e.g., a CAS of 4, 5, 6, 7, 8, 9 or 10.

In certain other embodiments, a patient to be treated according to the methods of the invention has or is diagnosed with inactive thyroid eye disease. Thus, the present invention also includes methods of treating inactive and/or chronic thyroid eye disease in a patient in need thereof. Inactive TED refers to the phases of the disease where the inflammatory aspects are less pronounced, but the patient is still experiencing symptoms affecting their quality of life, including proptosis and/or diplopia, arising from the continued tissue expansion and fibrosis behind the eyes. Inactive TED can be diagnosed using the CAS (either the 7-component or 10-component scale). A CAS of ≤2/7 on the on the 7-component scale and a CAS of ≤3/10 on the 10-component scale can be used to diagnose inactive TED in a patient. In certain embodiments, the patient with inactive TED to be treated according to the methods of the invention has a CAS of <2 on the 7-component scale (i.e. a CAS of 0 to 2) in both eyes. In other embodiments, the patient with inactive TED to be treated according to the methods of the invention has a CAS of ≤3 on the 10-component scale (i.e. a CAS of 0 to 3) in both eyes. In yet other embodiments, the patient with inactive TED to be treated according to the methods of the invention has a CAS of 0 or 1 in at least one eye on either the 7-component or 10-component scale. In still other embodiments, the patient with inactive TED to be treated according to the methods of the invention has a CAS of 0 or 1 in both eyes on either the 7-component or 10-component scale.

In certain embodiments, described herein is a method of treating an individual or patient with inactive and/or chronic thyroid eye disease, the method comprising administering to an individual or patient with inactive and/or chronic thyroid eye disease (TED) an IGF-1R antagonist antibody as described herein, thereby treating the inactive and/or chronic TED. In some embodiments, the individual or patient with inactive TED has a CAS of 2 or less. In other embodiments, the individual or patient with inactive TED has a CAS of 1 or less. In certain embodiments, the individual or patient with inactive TED has a CAS of 0. In certain other embodiments, the methods used to treat inactive TED may in certain instances be used in patients or individuals if both eyes have a CAS of lower than 2, e.g. 2, 1 or 0. In some embodiments, the individual with inactive TED has previously been treated when exhibiting an active disease state. In some embodiments, the TED has been inactive for at least 1, 2, 3, 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 48, 50 or 60 months.

TED has also been categorized into acute or chronic TED based on duration of the disease. Acute TED has been considered to be a disease duration of less than about 12 months, whereas chronic TED is typically considered to have a disease duration greater than 12 months. The understanding of the natural history of TED has evolved in recent years and evidence indicates that TED should be viewed as a progressive, heterogenous, autoimmune disease with a patient experiencing both active and inactive disease periods throughout the course of the disease. For instance, some patients that may have chronic and inactive disease can experience a resurgence of active disease (e.g. a flare) at some point in their disease course. In certain embodiments, the TED has been chronic for at least 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 48, 50 or 60 months or more. In certain other embodiments, the TED has been chronic and inactive for at least 4, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 48, 50 or 60 months. In certain embodiments, the individual or patient to be treated according to the methods of the invention has both inactive and chronic TED.

In some embodiments, patients to be treated according to the methods of the invention have or are diagnosed with moderate to severe thyroid eye disease. As described above, patients with moderate to severe thyroid eye disease usually have any one or more of the following: lid retraction ≥2 mm, moderate or severe soft tissue involvement, proptosis ≥3 mm above normal for race and gender, and inconstant or constant diplopia (Gorman score 2-3). Such criteria can be assessed in a patient using any of the methods described above. For example, proptosis can be assessed using an exophthalmometer or through CT or MRI imaging. Classification of the activity and severity of TED also takes into account the impact of TED symptoms on a patient's quality of life. Quality of life of a TED patient can be measured using self-assessment questionnaires, such as the Graves' ophthalmopathy quality of life (GO-QoL) questionnaire. See e.g., Terwee C B, Gerding M N, Dekker F W, Prummel M F, Wiersinga W M. Development of a disease specific quality of life questionnaire for patients with Graves' ophthalmopathy: the GO-QoL. Br J Ophthalmol. 1998 July; 82(7):773-9. In some embodiments, a patient or individual to be treated according to the methods of the invention has active and moderate to severe TED. In other embodiments, a patient or individual to be treated according to the methods of the invention has inactive and moderate to severe TED. In still other embodiments, a patient or individual to be treated according to the methods of the invention has chronic moderate to severe TED.

In certain embodiments, the methods described herein result in reduced proptosis, reduced diplopia, reduced orbital pain, reduced extraocular muscle volume, increased scores on the Graves' Ophthalmopathy Quality of Life (GO-QoL) questionnaire appearance and/or visual functioning subscales. In certain embodiments, the methods described herein result in reduced proptosis in at least one eye of an individual or patient as compared to the individual's or patient's proptosis measurement prior to administration of the IGF-1R antagonist antibody (i.e. pre-treatment baseline) or as compared to the proptosis measurement of a patient or individual not receiving the IGF-1R antagonist antibody. In certain other embodiments, the methods described herein result in reduced orbital pain in at least one eye of an individual or patient as compared to the individual's or patient's level of orbital pain prior to administration of the IGF-1R antagonist antibody (i.e. pre-treatment baseline) or as compared to the level of orbital pain of a patient or individual not receiving the IGF-1R antagonist antibody. In some embodiments, the methods described herein result in reduced extraocular muscle volume as compared to the extraocular muscle volume of the patient or individual prior to administration of the IGF-1R antagonist antibody (i.e. pre-treatment baseline) or as compared to the extraocular muscle volume of a patient or individual not receiving the IGF-1R antagonist antibody. In other embodiments, the methods described herein result in increased scores on the Graves' Ophthalmopathy Quality of Life (GO-QoL) questionnaire appearance and/or visual functioning subscales as compared to the patient's or individual's scores prior to administration of the IGF-1R antagonist antibody (i.e. pre-treatment baseline) or as compared to the scores of a patient or individual not receiving the IGF-1R antagonist antibody. In certain embodiments, the methods described herein result in increased scores on the Graves' Ophthalmopathy Quality of Life (GO-QoL) questionnaire appearance and/or visual functioning subscales by at least 5, 6, 7, 8, 9, 10, 11, 12, 13, 14, 15, 16, 17, 18, 19, or 20 or more points.

In certain embodiments of the methods of the invention, administration of any of the IGF-1R antagonist antibodies described herein reduces proptosis a patient afflicted with TED (e.g. active TED, inactive TED, or chronic TED). In some embodiments, administration of the IGF-1R antagonist antibodies described herein reduce proptosis by at least about 1 mm in the patient afflicted with TED (e.g. active, inactive, or chronic TED). In other embodiments, administration of the IGF-1R antagonist antibodies described herein reduce proptosis by at least about 2 mm in the patient afflicted with TED (e.g. active, inactive, or chronic TED). In certain embodiments, administration of the IGF-1R antagonist antibodies described herein reduce proptosis by at least about 3 mm in the patient afflicted with TED (e.g. active, inactive, or chronic TED). In certain other embodiments, administration of the IGF-1R antagonist antibodies described herein reduce proptosis by at least about 4 mm in the patient afflicted with TED (e.g. active, inactive, or chronic TED). In certain embodiments, patients or individuals to be treated according to the methods of the invention have an increase in proptosis of 3 mm or more in at least one eye prior to administration of the IGF-1R antagonist antibody (i.e. at baseline). The increase in proptosis can be relative to the patient's or individual's prior measurements (e.g. prior to diagnosis of TED) or relative to the normal average for the patient's or individual's race and gender. In certain other embodiments, patients or individuals to be treated according to the methods of the invention have a proptosis measurement of at least 18 mm prior to administration of the IGF-1R antagonist antibody (i.e. at baseline).

In some embodiments of the methods of the invention, administration of the IGF-1R antagonist antibodies described herein reduces the severity of diplopia in the patient or individual by ≥1 grade, ≥2 grades, or ≥3 grades on the Gorman scale in a patient afflicted with TED (e.g. active, inactive, or chronic TED) as compared to the patient's or individual's degree of diplopia prior to administration of the IGF-1R antagonist antibody (i.e. pre-treatment baseline) or as compared to the degree of diplopia of a patient or individual not receiving the IGF-1R antagonist antibody. In some cases, administration of the IGF-1R antagonist antibodies described herein reduces diplopia to a grade of 0 in a patient afflicted with TED (e.g. active, inactive, or chronic TED)—i.e. completely resolves diplopia in the patient. In certain embodiments, administration of the IGF-1R antagonist antibodies described herein reduces diplopia by 10%, 20%, 30%, 40%, 50% or more in a patient afflicted with TED (e.g. active, inactive, or chronic TED). In certain other embodiments, administration of the IGF-1R antagonist antibodies described herein completely resolves diplopia in the patient or individual. In certain embodiments, including any of the foregoing embodiments, the patients or individuals to be treated according to the methods of the invention have inconstant or constant diplopia prior to administration of the IGF-1R antagonist antibody (i.e. at baseline).

In certain embodiments of the methods of the invention, administration of the IGF-1R antagonist antibodies described herein reduces binocular diplopia in a patient afflicted with TED (e.g. active, inactive, or chronic TED). Binocular diplopia occurs when both eyes are open and resolves when either eye is closed. It is caused by a misalignment of the eyes, also called strabismus. Diplopia, including binocular diplopia, can be measured using a diplopia score that ranges from 0-3 using the Gorman scale as described in detail above. In some cases, a reduction of ≥1 grade indicates a patient is responsive. In some cases, a binocular diplopia score is based on measurements taken from both eyes simultaneously. In some cases, a binocular diplopia score is based on measurements taken from each eye separately. In this latter case, the binocular diplopia score can be calculated based on the average score of both eyes.

In some embodiments, administration of the IGF-1R antagonist antibodies described herein reduces binocular diplopia by ≥1 grade, ≥2 grades, or ≥3 grades in a patient afflicted with TED (e.g. active, inactive, or chronic TED). In certain embodiments, administration of the IGF-1R antagonist antibodies described herein reduces binocular diplopia to a grade of 0 in a patient afflicted with TED (e.g. active, inactive, or chronic TED). In certain other embodiments, administration of the IGF-1R antagonist antibodies described herein reduces binocular diplopia by 10%, 20%, 30%, 40%, 50% or more in a patient afflicted with TED (e.g. active, inactive, or chronic TED). In some embodiments, administration of the IGF-1R antagonist antibodies described herein completely resolves binocular diplopia in the patient. In some embodiments, the diplopia is constant diplopia. In some embodiments, the diplopia is inconstant diplopia. In some embodiments, the diplopia is intermittent diplopia.

In some embodiments, the improvement in or reduction in severity of diplopia is sustained for at least 20, 30, 40, or 50 weeks after discontinuation of administration of the IGF-1R antagonist antibody. In certain embodiments, the improvement in or reduction in severity of diplopia is sustained 20-30, 30-40, 40-50, or 50-60 weeks after discontinuation of administration of the IGF-1R antagonist antibody. In some embodiments, the improvement in or reduction in severity of diplopia is sustained for at least 20 weeks after discontinuation of administration of the IGF-1R antagonist antibody. In other embodiments, the improvement in or reduction in severity of diplopia is sustained for at least 50 weeks after discontinuation of administration of the IGF-1R antagonist antibody.

### Pharmaceutical Compositions

In certain embodiments of the methods of the invention, the IGF-1R antagonist antibodies described herein are administered to the patient or individual with TED in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

Antibodies in formulations for subcutaneous administration are generally present in highly concentrated form comprising greater than 50 mg/ml, 100 mg/ml, 200 mg/ml, or 300 mg/ml. Many excipients useful for subcutaneous formulations are known. See. e.g., Wang et al., Antibody Therapeutics, 2021, Vol. 4, No. 4 262-273.

In some embodiments, pharmaceutical compositions for use in the methods of the invention comprise any one of the IGF-1R antagonist antibodies described herein (e.g. about 130 mg/mL to about 170 mg/mL of an IGF-1R antagonist antibody), about 15 mM to about 25 mM histidine (e.g. histidine/histidine hydrochloride), about 200 mM to about 275 mM trehalose, about 30 mM to about 50 mM methionine, and about 0.05% (w/v) to about 0.35% (w/v) poloxamer 188. The pH of these formulations is in the range of about 5.0 to about 6.0 (e.g., pH of about 5.0, about 5.1, about 5.2, about 5.3, about 5.4, about 5.5, about 5.6, about 5.7, about 5.8, about 5.9, or about 6.0). In one particular embodiment, the pharmaceutical composition comprises about 130 mg/mL to about 170 mg/mL of an IGF-1R antagonist antibody described herein, about 20 mM histidine (e.g. histidine/histidine hydrochloride), about 210 mM trehalose, about 40 mM methionine, and about 0.2% (w/v) poloxamer 188, wherein the pharmaceutical composition has a pH of about 5.5±0.5.

In certain embodiments, the IGF-1R antagonist antibodies described herein can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the antibodies described herein in a suitable container and one or more additional components selected from: instructions for use; a diluent, an excipient, a carrier, and a device for administration (e.g., a syringe/needle or other injector).

In certain embodiments, described herein is a method of preparing a composition for inhibiting IGF-1R signaling (e.g. to treat TED or cancer) in an individual comprising admixing one or more pharmaceutically acceptable excipients, carriers, or diluents and an IGF-1R antagonist antibody described herein. In certain embodiments, described herein is a method of preparing a cancer treatment for storage or shipping comprising lyophilizing one or more antibodies of the current disclosure.

The following are additional embodiments of the invention for exemplary purposes and are not intended to be limiting in any way.

Embodiment 1. A method of treating thyroid eye disease (TED) in an individual in need thereof, comprising administering to the individual an effective amount of an antibody or antigen binding fragment thereof that binds insulin-like growth factor 1 receptor (IGF-1R), wherein the antibody or antigen binding fragment thereof comprises:
(a) an immunoglobulin heavy chain CDR1 (HCDR1) comprising the amino acid sequence SX₁GMH (SEQ ID NO: 71), wherein X₁ is H, Y, A, or T;
(b) an immunoglobulin heavy chain CDR2 (HCDR2) comprising the amino acid sequence X₁IX₂X₃DX₄SX₅TYYADSVRG (SEQ ID NO: 72), wherein X₁ is I, T, or Y; X₂ is W, N, or A; X₃ is F, H, A, or G; X₄ is G or A; and X₅ is S or T;
(c) an immunoglobulin heavy chain CDR3 (HCDR3) comprising the amino acid sequence ELX₁RRYFDL (SEQ ID NO: 73), wherein X₁ is G or N;
(d) an immunoglobulin light chain CDR1 (LCDR1) comprising the amino acid sequence RASQSVSSX₁LA (SEQ ID NO: 74), wherein X₁ is Y, A, or T;
(e) an immunoglobulin light chain CDR2 (LCDR2) comprising the amino acid sequence DASKRAT (SEQ ID NO: 46); and
(f) an immunoglobulin light chain CDR3 (LCDR3) comprising the amino acid sequence QQRX₁KX₂PPWT (SEQ ID NO: 75), wherein X₁ is S or G; and X₂ is Y or W; wherein the antibody or antigen binding fragment thereof does not comprise an immunoglobulin heavy chain variable region identical to SEQ ID NO: 1 and/or an immunoglobulin light chain variable region identical to SEQ ID NO: 2, thereby treating the TED.

Embodiment 2. The method of Embodiment 1, wherein the amino acid residue corresponding to X₂ of the LCDR3 in SEQ ID NO: 75 is a tyrosine.

Embodiment 3. The method of Embodiments 1 or 2, wherein:
(a) the HCDR1 comprises the amino acid sequence of SEQ ID NO: 30 (SHGMH);
(b) the HCDR2 comprises the amino acid sequence of SEQ ID NO: 35 (YIWFDGSSTYYADSVRG);
(c) the HCDR3 comprises the amino acid sequence of SEQ ID NO: 41 (ELGRRYFDL);
(d) the LCDR1 comprises the amino acid sequence of SEQ ID NO: 43 (RASQSVSSALA);
(e) the LCDR2 comprises the amino acid sequence of SEQ ID NO: 46 (DASKRAT); and
(f) the LCDR3 comprises the amino acid sequence of SEQ ID NO: 48 (QQRSKYPPWT).

Embodiment 4. The method of Embodiments 1 or 2, wherein:
(a) the HCDR1 comprises the amino acid sequence of SEQ ID NO: 33 (SYGMH);
(b) the HCDR2 comprises the amino acid sequence of SEQ ID NO: 38 (IIWFDGSSTYYADSVRG);
(c) the HCDR3 comprises the amino acid sequence of SEQ ID NO: 41 (ELGRRYFDL);
(d) the LCDR1 comprises the amino acid sequence of SEQ ID NO: 45 (RASQSVSSYLA);
(e) the LCDR2 comprises the amino acid sequence of SEQ ID NO: 46 (DASKRAT);
and
(f) the LCDR3 comprises the amino acid sequence of SEQ ID NO: 48 (QQRSKYPPWT).

Embodiment 5. The method of Embodiments 1 or 2, wherein:
(a) the HCDR1 comprises the amino acid sequence of SEQ ID NO: 30 (SHGMH);
(b) the HCDR2 comprises the amino acid sequence of SEQ ID NO: 34 (IIAGDASTTYYADSVRG);
(c) the HCDR3 comprises the amino acid sequence of SEQ ID NO: 41 (ELGRRYFDL);
(d) the LCDR1 comprises the amino acid sequence of SEQ ID NO: 45 (RASQSVSSYLA);
(e) the LCDR2 comprises the amino acid sequence of SEQ ID NO: 46 (DASKRAT); and
(f) the LCDR3 comprises the amino acid sequence of SEQ ID NO: 48 (QQRSKYPPWT).

Embodiment 6. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 3; and wherein the immunoglobulin light chain variable region comprises an amino acid sequence at least about 90%, 95%, 97%, 99%, or 100% identical to that set forth in SEQ ID NO: 4.

Embodiment 7. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 5; and wherein the immunoglobulin light chain variable region comprises an amino acid sequence at least about 90%, 95%, 97%, 99%, or 100% identical to that set forth in SEQ ID NO: 6.

Embodiment 8. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 7; and wherein the immunoglobulin light chain variable region comprises an amino acid sequence at least about 90%, 95%, 97%, 99%, or 100% identical to that set forth in SEQ ID NO: 8.

Embodiment 9. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 9; and wherein the immunoglobulin light chain variable region comprises an amino acid sequence at least about 90%, 95%, 97%, 99%, or 100% identical to that set forth in SEQ ID NO: 10.

Embodiment 10. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 11; and wherein the immunoglobulin light chain variable region comprises an amino acid sequence at least about 90%, 95%, 97%, 99%, or 100% identical to that set forth in SEQ ID NO: 12.

Embodiment 11. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 13; and wherein the immunoglobulin light chain variable region comprises an amino acid sequence at least about 90%, 95%, 97%, 99%, or 100% identical to that set forth in SEQ ID NO: 14.

Embodiment 12. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 15; and wherein the immunoglobulin light chain variable region comprises an amino acid sequence at least about 90%, 95%, 97%, 99%, or 100% identical to that set forth in SEQ ID NO: 16.

Embodiment 13. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 17; and wherein the immunoglobulin light chain variable region comprises an amino acid sequence at least about 90%, 95%, 97%, 99%, or 100% identical to that set forth in SEQ ID NO: 18.

Embodiment 14. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 19; and wherein the immunoglobulin light chain variable region comprises an amino acid sequence at least about 90%, 95%, 97%, 99%, or 100% identical to that set forth in SEQ ID NO: 20.

Embodiment 15. The method of Embodiment 1, comprising an immunoglobulin heavy chain variable region and an immunoglobulin light chain variable region, wherein the immunoglobulin heavy chain variable region comprises an amino acid sequence at least about 90%, 95%, 97%, 99%, or 100% identical to that set forth in SEQ ID NO: 21; and wherein the immunoglobulin light chain variable region comprises an amino acid sequence at least about 90%, 95%, 97%, 99%, or 100% identical to that set forth in SEQ ID NO: 22.

Embodiment 16. The method of Embodiment 1, comprising an immunoglobulin heavy chain and an immunoglobulin light chain, wherein the immunoglobulin heavy chain comprises an amino acid sequence at least about 90%, 95%, 97%, 99%, or 100% identical to that set forth in SEQ ID NO: 51; and wherein the immunoglobulin light chain comprises an amino acid sequence at least about 90%, 95%, 97%, 99%, or 100% identical to that set forth in SEQ ID NO: 52.

Embodiment 17. The method of any one of Embodiments 1 to 16, wherein the antibody or antigen binding fragment thereof is an IgG antibody or antigen binding fragment thereof.

Embodiment 18. The method of any one of Embodiments 1 to 16, wherein the antigen binding fragment comprises a Fab, F(ab)₂, or a single chain variable fragment (scFv).

Embodiment 19. The method of any one of Embodiments 1 to 18, wherein the antibody or antigen binding fragment thereof is chimeric or humanized.

Embodiment 20. The method of any one of Embodiments 1 to 17 and 19, wherein the antibody comprises M252Y, S254T, and T256E substitutions according to EU numbering in one or both heavy chain constant regions.

Embodiment 21. The method of any one of Embodiments 1 to 20, wherein the antibody or antigen binding fragment thereof is characterized by a half-life of 25 days or longer in a human.

Embodiment 22. The method of any one of Embodiments 1 to 20, wherein the antibody or antigen binding fragment thereof is characterized by a half-life of 30 days or longer in a human.

Embodiment 23. The method of any one of Embodiment 1 to 22, wherein the antibody or antigen binding fragment inhibits signaling through IGF-1R.

Embodiment 24. The method of any one of Embodiment 1 to 23, wherein the antibody or antigen binding fragment inhibits phosphorylation of IGF-1R with an EC50 of 10 ng/mL or less.

Embodiment 25. The method of any one of Embodiments 1 to 23, wherein the antibody or antigen binding fragment inhibits phosphorylation of IGF-1R with an EC50 of 9 ng/mL or less.

Embodiment 26. The method of any one of Embodiments 1 to 23, wherein the antibody or antigen binding fragment inhibits phosphorylation of IGF-1R with an EC50 of 7 ng/mL or less.

Embodiment 27. The method of any one of Embodiments 1 to 26, wherein the antibody or antigen binding fragment binds to the IGF-1R with a K_{D} of less than 5×10⁻⁹ M.

Embodiment 28. The method of any one of Embodiments 1 to 26, wherein the antibody or antigen binding fragment binds to the IGF-1R with a K_{D} of less than 1×10⁻⁹ M.

Embodiment 29. The method of any one of Embodiments 1 to 26, wherein the antibody or antigen binding fragment binds to the IGF-1R with a K_{D} of less than 5×10⁻¹⁰ M.

Embodiment 30. The method of any one of Embodiments 1 to 29, wherein the antibody or antigen binding fragment thereof is formulated for intravenous administration.

Embodiment 31. The method of any one of Embodiments 1 to 29, wherein the antibody or antigen binding fragment thereof is formulated for subcutaneous administration.

Embodiment 32. The method of any one of Embodiments 1 to 31, wherein the method reduces proptosis by at least 2 mm in the individual in need thereof.

Embodiment 33. The method of Embodiment 32, wherein proptosis is reduced by at least 3 mm.

Embodiment 34. The method of Embodiment 33, wherein proptosis is reduced by at least 4 mm.

Embodiment 35. The method of any one of Embodiments 1 to 34, wherein the method reduces the clinical activity score (CAS) of the individual.

Embodiment 36. The method of Embodiment 35, wherein the CAS is reduced by at least 2 points.

Embodiment 37. The method of Embodiment 36, wherein the CAS is reduced by at least 3 points.

Embodiment 38. The method of any one of Embodiments 35 to 37, wherein the CAS of the individual in need thereof is reduced to one or less.

Embodiment 39. The method of any one of Embodiments 35 to 37, wherein the CAS of the individual in need thereof is reduced to zero.

Embodiment 40. The method of any one of Embodiments 1 to 39, wherein the method reduces the severity of diplopia in the individual in need thereof.

Embodiment 41. The method of Embodiment 40, wherein the diplopia is constant diplopia.

Embodiment 42. The method of Embodiment 40, wherein the diplopia is intermittent diplopia.

Embodiment 43. The method of Embodiment 40, wherein the diplopia is inconstant diplopia.

Embodiment 44. The method of any one of Embodiments 40 to 43, wherein the reduction in the severity of diplopia is sustained at least 20 weeks after discontinuation of the antibody or antigen binding fragment thereof.

Embodiment 45. The method of any one of Embodiments 40 to 43, wherein the improvement in or reduction in severity of diplopia is sustained at least 50 weeks after discontinuation of inhibitor administration.

Embodiment 46. The method of any one of Embodiment 1 to 45, wherein the method improves quality of life in the individual in need thereof.

Embodiment 47. The method of Embodiment 46, wherein the quality of life is measured by the Graves' Ophthalmopathy Quality of Life (GO-QoL) assessment, on either the visual functioning subscale or appearance subscale thereof.

Embodiment 48. The method of Embodiment 47, wherein the GO-QoL is improved by at least 8 points.

Embodiment 49. The method of Embodiment 47, wherein the visual functioning subscale is improved.

Embodiment 50. The method of Embodiment 47, wherein the appearance subscale is improved.

Embodiment 51. The method of any one of Embodiments 1 to 50, wherein the TED is moderate-to-severe TED.

Embodiment 52. The method of any one of Embodiments 1 to 51, wherein the TED is active/acute TED.

Embodiment 53. The method of any one of Embodiments 1 to 52, wherein the TED is inactive/chronic TED.

### EXAMPLES

The following illustrative examples are representative of embodiments of compositions and methods described herein and are not meant to be limiting in any way.

### Example 1—Determination of Antibody Affinity

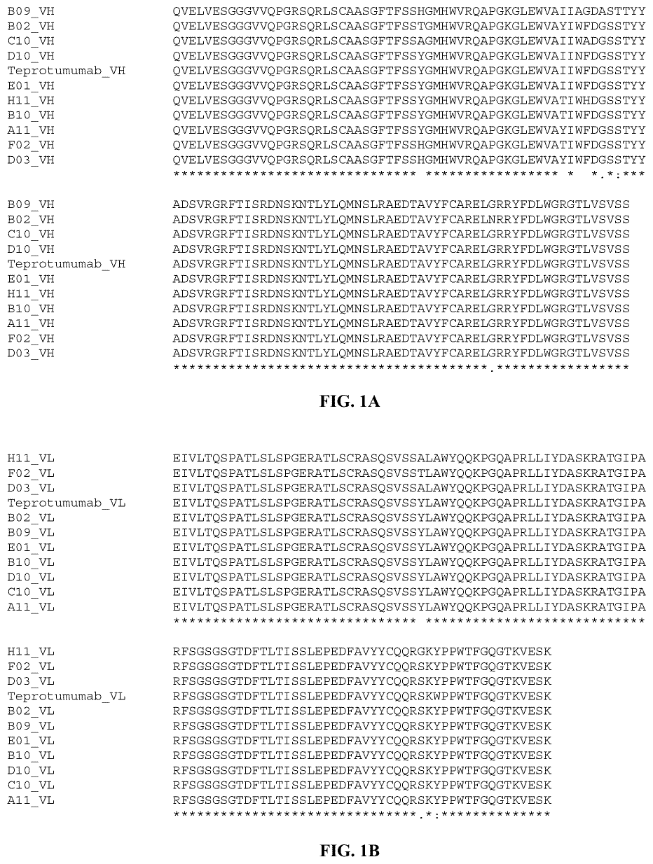

In order to develop antibodies with higher affinities needed for a subcutaneous formulation multiple mutants of a reference antibody with teprotumumab variable regions (comprising variable regions comprising SEQ ID NO: 1 and SEQ ID NO: 2) were tested to determine binding affinity. The antibodies described herein were tested for binding affinity to IGF-1R by Octet. Results are shown below in Table 1. Sequence alignments of all teprotumumab mutants are shown in FIG. 1A and FIG. 1B. Surprisingly, mutation of the tryptophan at position 94 of the light chain to tyrosine was present in all antibodies. This was surprising as generally light chain CDRs are less important to binding affinity than heavy chain CDRs.

TABLE 1

| Antibody (Fab) | K_{D} (M) | Kₒₙ (1/Ms) | K_{off} (1/s) |
|---|---|---|---|
| Reference | 5.17E−09 | 1.51E+06 | 7.81E−03 |
| (Teprotumumab) | | | |
| B10 | 8.98E−10 | 9.21E+05 | 8.26E−04 |
| B09 | 1.13E−09 | 7.05E+05 | 7.99E−04 |
| B02 | 1.54E−09 | 8.10E+05 | 1.25E−03 |
| D10 | 6.90E−10 | 9.84E+05 | 6.79E−04 |
| F02 | 1.92E−09 | 1.20E+06 | 2.30E−03 |
| H11 | 4.18E−10 | 1.78E+06 | 7.44E−04 |
| C10 | 4.47E−10 | 1.08E+06 | 4.85E−04 |
| D03 | 4.07E−10 | 1.48E+06 | 6.03E−04 |
| A11 | 8.49E−10 | 8.95E+05 | 7.60E−04 |
| E01 | 1.79E−10 | 2.66E+06 | 2.98E−04 |

### Affinity Measurements of Teprotumumab Variants Via Biolayer Inferometry

Human IGF-1R (Glu 31-Asn 932 with a polyhistidine tag at the C-terminus) expressed from human 293 cells (HEK293) was purchased from ACROBiosystems (Newark, DE). Dip and Read Ni-NTA (NTA) Biosensors from Sartorius (Bohemia, NY) pre-immobilized with nickel-charged Tris-NTA, were enabled for kinetic characterization of novel anti-human IGF-1R antibodies. Briefly, antibody variants were digested into purified Fab fragments (to enable 1:1 binding and global fitting). The FabALACTICA Fab kit from GENOVIS (Cambridge, MA) was used to generate a monovalent binding Fab domain of each antibody variant, Fab fragments are subsequently separated from Fe using the CaptureSelect™ Fc column. Binding of variant Fabs to NTA-captured human IGF-1R (His-tagged) was monitored by biolayer interferometry (BLI) on an Octet RED96e (Sartorius). NTA biosensors were charged with 10 mM NiCl2, then loaded with His-tagged human IGF-IR at approximately 5 μg/mL to an average loading response of 0.67 nm shift. Variant Fabs were assayed from 0-100 nM in 10× kinetics buffer (1×PBS, 0.1% BSA, 0.02% Tween-20 plus Kathon as a preservative) from Sartorius. Binding kinetics and affinities were determined using the Octet Analysis Studio Software (Sartorius) by applying a 1:1 global fitting to double-referenced subtracted data.

### Example 2—Biological Activity of Teprotumumab Mutants

The biological activity of teprotumumab mutants was determined by assessing inhibition of IGF-1R phosphorylation and binding of ligands IGF-1 and IGF-2 to the receptor. Results are shown in Tables 2 to 4. Overall clones showed biological activity comparable to or better than the reference teprotumumab, indicating that improvements in affinity seen in example 1 extend to biological activity as well.

TABLE 2
Inhibition of Phosphorylation

| | IC50 (ng/mL) |
|---|---|
| E01 | 6.4 |
| D03 | 4.9 |
| D10 | 6.7 |
| B09 | 8.3 |
| B10 | 16.2 |
| B02 | 13.5 |
| H11 | 21.1 |
| C10 | 6.5 |
| A11 | 11.5 |
| Teprotumumab reference | 10.6 |

TABLE 3
IGF-1 inhibition

| | IC50 (ng/mL) |
|---|---|
| E01 | 387.5 |
| D03 | 387.8 |
| D10 | 386.3 |
| B09 | 336.8 |
| B10 | 394.5 |
| B02 | 385.5 |
| H11 | 452 |
| C10 | No data |
| A11 | 351.5 |
| Teprotumumab reference | 449 |

TABLE 4
IGF-2 inhibition

| | IC50 (ng/mL) |
|---|---|
| E01 | 141.5 |
| D03 | 160.5 |
| D10 | 173.5 |
| B09 | 153.5 |
| B10 | 176.0 |
| B02 | 114.5 |
| H11 | 156.5 |
| C10 | No data |
| A11 | 157.5 |
| Teprotumumab reference | 260.3 |

Inhibition of IGF-1R phosphorylation

4×10\hat{ }4 NCI-H322 cells/well were seeded in flat bottom 96 well plate and incubated overnight in a 37° C., 5% incubator. Cells were treated with different concentration of anti-IGF1R for 1 hour and stimulated with 200 ng/mL of recombinant human IGF-1 protein for 30 minutes. Then, the phosphorylated IGF-1 in cell lysate was determined by Insulin Signaling Panel whole cell lysate kit (MSD) following manufactural protocol.

### IGF-1 Inhibition of Binding Assay

Maxisorp plates were coated with 1.5 ug/mL of IGF-1R overnight at 4° C. Plates were washed and blocked with 1% BSA. Subsequently, serially diluted anti-IGF1R antibodies were incubated for 30 min at room temperature followed by an incubation with a final concentration of 20 ng/mL IGF-1 biotin. Plates were washed and streptavidin-horseradish peroxidase was added. After a final wash, 3,3′,5,5′-Tetramethylbenzidine was added for 8 minutes. The reaction was terminated with a HCL based stop solution. Plates were measured for absorbance at 450 nM, and the data was analyzed using Softmax Pro 7.1 software.

### IGF-2 Inhibition of Binding Assay

Maxisorp plates were coated with 1.5 ug/mL of IGF-1R overnight at 4° C. Plates were washed and blocked with 1% BSA. Subsequently, serially diluted anti-IGF1R antibodies were incubated for 30 min at room temperature followed by an incubation with a final concentration of 200 ng/mL of IGF-2 biotin. Plates were washed and streptavidin-horseradish peroxidase was added. After a final wash, 3,3′,5,5′-Tetramethylbenzidine was added for 8 minutes. The reaction was terminated with a HCL based stop solution. Plates were measured for absorbance at 450 nM, and the data was analyzed using Softmax Pro 7.1 software.

### Example 3—Clone D03 with YTE Mutation Shows ADCC Activity Comparable to Teprotumumab

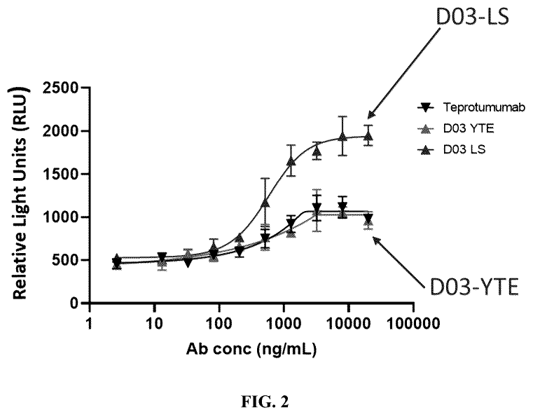

Higher affinity antibodies may promote greater antibody-dependent cell cytotoxicity (ADCC) when administered to an individual. For use in treating ophthalmic conditions and other conditions associated with autoimmune or inflammatory conditions, an increase in ADCC would be undesirable. Clone D03 was selected for further testing of its ADCC activity using different heavy chain constant region mutations (the “YTE” mutation, which possesses mutations at M252Y/S254T/T256E according to EU numbering; and the “LS” mutation Met428Leu/Asn434Ser according to EU numbering). As shown in FIG. 2, compared to teprotumumab (inverted triangles), clone D03 formatted with an LS (“D03-LS”) mutation surprisingly showed increased ADCC compared to clone D03 with the YTE mutation (“D03-YTE”). D03-YTE showed ADCC comparable to teprotumumab. This finding was surprising as the YTE mutations have been reported to reduce affinity to FcγRIIIA and significantly reduce ADCC activity (see Dall'Acqua et al., J. Biol. Chem., Vol. 281: 23514-23524, 2006), whereas the LS mutations have been reported to have no effect on ADCC activity (see, e.g., Saunders, Front Immunol., Vol. 10:1296, 2019).

### ADCC Assay

Antibodies were incubated with DU145 cells for 4 hours at the indicated concentration, at which time effector cells with a luminescent ADCC reporter were added for overnight incubation, BioGlo detection reagent was added for 10 minutes and results were read on a luminometer.

### Example 3—Clone D03 with YTE Mutation Exhibits 2-3 Fold Increased Half-Life Compared to Teprotumumab

The half-life of clone D03-YTE was tested in cynomolgus monkeys. Cynomolgus monkeys were dosed with D03-YTE at 150 mg/kg IV, 150 mg/kg subcutaneous, or 75 mg/kg subcutaneous. Serum samples were collected at 2, 6, 24, 96, 168, 336, 504, 672, 1008, 1344, 1680, and 2016 hours. Serum samples were analyzed for D03-YTE by ELISA. As shown below in Table 5 D03 possesses an increased serum half-life compared to teprotumumab.

TABLE 5

| | | | | AUC_{inf} | | |
|---|---|---|---|---|---|---|
| Dose (mg/kg) | | Route | t_{1/2} (day)¹ | (μg · day/mL) | Cₘₐₓ (μg/mL) | |
| 150 | (n = 3) | IV | 29.9 (4.6) | 47980 (5941) | 3321 | (489) |
| 150 | (n = 2)² | SC | 22.4 | 27562 | 804 | (33.0) |
| 75 | (n = 5)² | SC | 18.6 (3.2) | 12905 (2001) | 412 | (48.4) |

| | | | | |
|---|---|---|---|---|
| Tepro (150) | IV | 10.1 | NA | NA |

### Example 4—Clone D03 with YTE Mutation Exhibits Increased Inhibition of IGF-1R Phosphorylation Compared to Teprotumumab

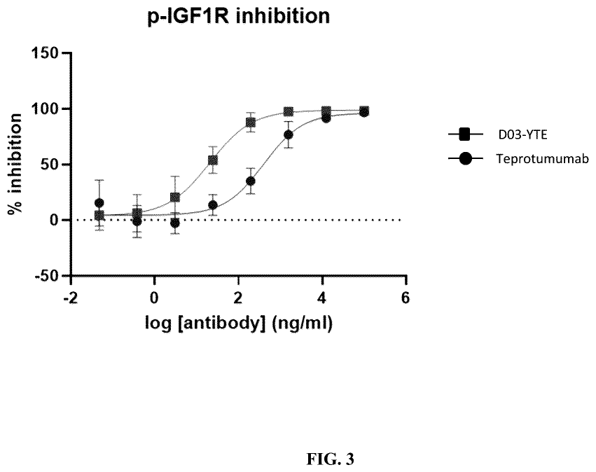

D03-YTE was tested for its ability to inhibit IGF-1R phosphorylation. Experiments as described in Example 2 were conducted, with the following modification: dilution of anti-IGF-1R antibodies were started at 100 μg/mL and diluted 1/8 allowing 100% inhibition by teprotumumab. The experiments in FIG. 3 show that in this experimental set up Teprotumumab (circles) exhibited an IC50 of 397.4 ng/mL vs. 20.51 ng/mL for D03-YTE (squares).

### Example 5—Teprotumumab Mutants Retain the Manufacturability of the Parent Clone

One limiting factor when developing antibodies for subcutaneous injection, is that in addition to binding with high-affinity and/or possessing longer half-lives in vivo, the antibodies must possess favorable biophysical properties such as low hydrophobicity, propensity to form aggregates, and the ability to exist in formulations with a low viscosity. Any mutation made to antibody has the potential to negatively affect these characteristics, however as shown in this example despite increased affinity and biological potency there were no deleterious changes with respect to key manufacturability criteria. As shown in Table 6 D03 with a YTE mutation exhibited no increase in degradation when subjected to forced degradation at 40° C. for 28 days when compared to teprotumumab with a YTE mutation (Buffer composition: 20 mM Histidine/Histidine-HCl, 40 mM L-Methionine, 210 mM Trehalose, pH 5.5, 0.2% PX188).

TABLE 6

| | | | Fragmentation/ | Aggregation/ |
|---|---|---|---|---|
| | % Monomer loss/Month | | Month | month |
| | No | T0 | No | T0 |
| | exclusions | Excluded | exclusions | Excluded |
| B09 LS | 0.4 | 0.52 | 0.52 | −0.16 |
| B09 YTE | 0.6 | 0.68 | 0.6 | 0 |
| D03 LS | 0.24 | 0.4 | 0.52 | −0.24 |
| D03 YTE | 0.4 | 0.4 | 0.52 | −0.16 |
| E01 LS | 0.4 | 0.52 | 0.52 | −0.12 |
| E01 YTE | 0.48 | 0.52 | 0.52 | 0 |
| Tepro LS | N.A. | 0.28 | 0.6 | −1.04 |
| Tepro WT | 0 | 0.4 | 0.56 | −0.56 |
| Tepro YTE | 0.32 | 0.52 | 0.64 | −0.24 |

Anti-IGF-1R variants were assessed to ensure that Fc and variable domain mutations did not significantly increase viscosity. See Table 7. Increased viscosity is undesirable as it may limit liquid formulation options for subcutaneous administration. Viscosity of the anti-IGF-1R variants were initially assessed at −130 mg/ml. One variant (B09-YTE) exhibited increased viscosity compared to other variants and control mAbs and this variant was not assessed at higher concentrations. As there was little differentiation at −130 mg/ml, viscosity was assessed at a higher concentration (˜170 mg/ml). At 170 mg/ml all D03 and E01 variants had viscosity measurements below 20 cP and were similar to teprotumumab controls.

Antibodies were formulated in the following buffer: 20 mM Histidine/Histidine-HCl, 40 mM L-Methionine, 210 mM Trehalose, pH 5.5, 0.2% PX188. Antibodies were evaluated at 130 mg/ml & 170 mg/ml concentrations and at 20° & 25° C. Antibody concentration was determined via SoloVPE (in triplicate). Viscosity was assessed via a RheoSense m-VROC with the applying shear sweep rate 400-2700/s at 20° C. and 25° C. collecting 8-12 segments.

TABLE 7

| | Viscosity (cP) 135 mg/ml | | Viscosity (cP) 170 mg/ml | |
|---|---|---|---|---|
| | 20° C. | 25° C. | 20° C. | 25° C. |
| B09 LS | 8.1 | 6.9 | N.D. | N.D. |
| B09 YTE | 10.7 | 9.1 | N.D. | N.D. |
| DO3 LS | 8 | 6.3 | 15.1 | 11 |
| DO3 YTE | 8.4 | 6.6 | 12.7 | 9.3 |
| E01 LS | 7.8 | 6.5 | 12.8 | 10.5 |
| E01 YTE | 7.2 | 6 | 15.2 | 12.3 |
| Tepro LS | 7.2 | 6 | 13.9 | 11.2 |
| Tepro WT | 6.9 | 5.8 | 11.4* | 9.3* |
| Tepro YTE | 7.4 | 6.3 | N.D. | N.D. |

Anti-IGF-1R variants were assessed by Hydrophobic Interaction Chromatography (HIC) analysis (Table 8′). HIC is an emerging assessment of the hydrophobic character of antibodies and other proteins. Too much hydrophobic character can be detrimental to overall stability and lead to increases in non-specific binding and self-interactions. All Anti-IGF-1R variants had elution profiles similar to teprotumumab controls. No increases in hydrophobic character were observed. Teprotumumab and its variants eluted earlier that the positive control (NISTmAb) and significantly earlier than CNTO607 (a mAb with known hydrophobic character causing developability issues). These data suggest the mutations introduced to increase either IGF-1R affinity or half-life extension did not affect the overall hydrophobic character of the variants.

HIC was performed using an Agilent 1260 Infinity II HPLC with a ProPac HIC-10, 5 jam, 4.6×100 mm column (ThermoFisher PN:063655). Method parameters were taken directly from the MabPAC-10 product insert. The following buffers were used: BufferA: 2 M ammonium sulfate, 0.1 M sodium phosphate, 2-propanol (93:7 v/v), pH 7.0; BufferB: 0.1 M sodium phosphate, 2-propanol (93:7 v/v), pH 7.0. After 5 minutes equilibration in 95% Buffer A and 5% Buffer B, a 25-minute gradient ending with 100% buffer B was employed. The temperature was 30° C. and UV detection at 214 nM was used to visualize protein elution. NISTmAb (positive control human IgG1) and CNTO607 (a mAb with known hydrophobic character) were also evaluated as comparators.

TABLE 8

| | Retention Time (min) |
|---|---|
| B09 LS | 21.0 |
| B09 YTE | 20.9 |
| D03 LS | 21.2 |
| D03 YTE | 21.1 |
| E01 LS | 21.2 |
| E01 YTE | 21.0 |
| Tepro LS | 21.5 |
| Tepro WT | 21.3 |
| Tepro YTE | 21.4 |
| NISTmAb | 22.6 |
| CNTO607 | 25.7 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

TABLE 9
Sequence Listing

| SEQ ID | | |
|---|---|---|
| NO | Description | Amino Acid Sequence |
| 1 | Teprotumumab | QVELVESGGGVVQPGRSQRLSCAASGFTFSSYGMHWVRQAPG |
| | VH | KGLEWVAIIWFDGSSTYYADSVRGRFTISRDNSKNTLYLQMN |
| | | SLRAEDTAVYFCARELGRRYFDLWGRGTLVSVSS |
| 2 | Teprotumumab | EIVLTQSPATLSLSPGERATLSCRASQSVSSYLAWYQQKPGQ |
| | VL | APRLLIYDASKRATGIPARFSGSGSGTDFTLTISSLEPEDFA |
| | | VYYCQQRSKWPPWTFGQGTKVESK |
| 3 | B02 VH | QVELVESGGGVVQPGRSQRLSCAASGFTFSSTGMHWVRQAPG |
| | | KGLEWVAYIWFDGSSTYYADSVRGRFTISRDNSKNTLYLQMN |
| | | SLRAEDTAVYFCARELNRRYFDLWGRGTLVSVSS |
| 4 | B02 VL | EIVLTQSPATLSLSPGERATLSCRASQSVSSYLAWYQQKPGQ |
| | | APRLLIYDASKRATGIPARFSGSGSGTDFTLTISSLEPEDFA |
| | | VYYCQQRSKYPPWTFGQGTKVESK |
| 5 | B09 VH | QVELVESGGGVVQPGRSQRLSCAASGFTFSSHGMHWVRQAPG |
| | | KGLEWVAIIAGDASTTYYADSVRGRFTISRDNSKNTLYLQMN |
| | | SLRAEDTAVYFCARELGRRYFDLWGRGTLVSVSS |
| 6 | B09 VL | EIVLTQSPATLSLSPGERATLSCRASQSVSSYLAWYQQKPGQ |
| | | APRLLIYDASKRATGIPARFSGSGSGTDFTLTISSLEPEDFA |
| | | VYYCQQRSKYPPWTFGQGTKVESK |
| 7 | E01 VH | QVELVESGGGVVQPGRSQRLSCAASGFTFSSYGMHWVRQAPG |
| | | KGLEWVAIIWFDGSSTYYADSVRGRFTISRDNSKNTLYLQMN |
| | | SLRAEDTAVYFCARELGRRYFDLWGRGTLVSVSS |
| 8 | E01 VL | EIVLTQSPATLSLSPGERATLSCRASQSVSSYLAWYQQKPGQ |
| | | APRLLIYDASKRATGIPARFSGSGSGTDFTLTISSLEPEDFA |
| | | VYYCQQRSKYPPWTFGQGTKVESK |
| 9 | B10 VH | QVELVESGGGVVQPGRSQRLSCAASGFTFSSYGMHWVRQAPG |
| | | KGLEWVATIWFDGSSTYYADSVRGRFTISRDNSKNTLYLQMN |
| | | SLRAEDTAVYFCARELGRRYFDLWGRGTLVSVSS |
| 10 | B10 VL | EIVLTQSPATLSLSPGERATLSCRASQSVSSYLAWYQQKPGQ |
| | | APRLLIYDASKRATGIPARFSGSGSGTDFTLTISSLEPEDFA |
| | | VYYCQQRSKYPPWTFGQGTKVESK |
| 11 | D10 VH | QVELVESGGGVVQPGRSQRLSCAASGFTFSSYGMHWVRQAPG |
| | | KGLEWVAIINFDGSSTYYADSVRGRFTISRDNSKNTLYLQMN |
| | | SLRAEDTAVYFCARELGRRYFDLWGRGTLVSVSS |
| 12 | D10 VL | EIVLTQSPATLSLSPGERATLSCRASQSVSSYLAWYQQKPGQ |
| | | APRLLIYDASKRATGIPARFSGSGSGTDFTLTISSLEPEDFA |
| | | VYYCQQRSKYPPWTFGQGTKVESK |
| 13 | C10 VH | QVELVESGGGVVQPGRSQRLSCAASGFTFSSAGMHWVRQAPG |
| | | KGLEWVAIIWADGSSTYYADSVRGRFTISRDNSKNTLYLQMN |
| | | SLRAEDTAVYFCARELGRRYFDLWGRGTLVSVSS |
| 14 | C10 VL | EIVLTQSPATLSLSPGERATLSCRASQSVSSYLAWYQQKPGQ |
| | | APRLLIYDASKRATGIPARFSGSGSGTDFTLTISSLEPEDFA |
| | | VYYCQQRSKYPPWTFGQGTKVESK |
| 15 | A11 VH | QVELVESGGGVVQPGRSQRLSCAASGFTFSSYGMHWVRQAPG |
| | | KGLEWVAYIWFDGSSTYYADSVRGRFTISRDNSKNTLYLQMN |
| | | SLRAEDTAVYFCARELGRRYFDLWGRGTLVSVSS |
| 16 | A11 VL | EIVLTQSPATLSLSPGERATLSCRASQSVSSYLAWYQQKPGQ |
| | | APRLLIYDASKRATGIPARFSGSGSGTDFTLTISSLEPEDFA |
| | | VYYCQQRSKYPPWTFGQGTKVESK |
| 17 | D03 VH | QVELVESGGGVVQPGRSQRLSCAASGFTFSSHGMHWVRQAPG |
| | | KGLEWVAYIWFDGSSTYYADSVRGRFTISRDNSKNTLYLQMN |
| | | SLRAEDTAVYFCARELGRRYFDLWGRGTLVSVSS |
| 18 | D03 VL | EIVLTQSPATLSLSPGERATLSCRASQSVSSALAWYQQKPGQ |
| | | APRLLIYDASKRATGIPARFSGSGSGTDFTLTISSLEPEDFA |
| | | VYYCQQRSKYPPWTFGQGTKVESK |
| 19 | F02 VH | QVELVESGGGVVQPGRSQRLSCAASGFTFSSHGMHWVRQAPG |
| | | KGLEWVATIWFDGSSTYYADSVRGRFTISRDNSKNTLYLQMN |
| | | SLRAEDTAVYFCARELGRRYFDLWGRGTLVSVSS |
| 20 | F02 VL | EIVLTQSPATLSLSPGERATLSCRASQSVSSTLAWYQQKPGQ |
| | | APRLLIYDASKRATGIPARFSGSGSGTDFTLTISSLEPEDFA |
| | | VYYCQQRSKYPPWTFGQGTKVESK |
| 21 | H11 VH | QVELVESGGGVVQPGRSQRLSCAASGFTFSSYGMHWVRQAPG |
| | | KGLEWVATIWHDGSSTYYADSVRGRFTISRDNSKNTLYLQMN |
| | | SLRAEDTAVYFCARELGRRYFDLWGRGTLVSVSS |
| 22 | H11 VL | EIVLTQSPATLSLSPGERATLSCRASQSVSSALAWYQQKPGQ |
| | | APRLLIYDASKRATGIPARFSGSGSGTDFTLTISSLEPEDFA |
| | | VYYCQQRGKYPPWTFGQGTKVESK |
| 30 | HCDR1 | SHGMH |
| 31 | HCDR1 | STGMH |
| 32 | HCDR1 | SAGMH |
| 33 | HCDR1 | SYGMH |
| 34 | HCDR2 | IIAGDASTTYYADSVRG |
| 35 | HCDR2 | YIWFDGSSTYYADSVRG |
| 36 | HCDR2 | IIWADGSSTYYADSVRG |
| 37 | HCDR2 | IINFDGSSTYYADSVRG |
| 38 | HCDR2 | IIWFDGSSTYYADSVRG |
| 39 | HCDR2 | TIWHDGSSTYYADSVRG |
| 40 | HCDR2 | TIWFDGSSTYYADSVRG |
| 41 | HCDR3 | ELGRRYFDL |
| 42 | HCDR3 | ELNRRYFDL |
| 43 | LCDR1 | RASQSVSSALA |
| 44 | LCDR1 | RASQSVSSTLA |
| 45 | LCDR1 | RASQSVSSYLA |
| 46 | LCDR2 | DASKRAT |
| 47 | LCDR3 | QQRGKYPPWT |
| 48 | LCDR3 | QQRSKYPPWT |
| 49 | LCDR3 | QQRSKWPPWT |
| 51 | D03 Heavy | QVELVESGGGVVQPGRSQRLSCAASGFTFSSHGMHWVRQAPG |
| | chain | KGLEWVAYIWFDGSSTYYADSVRGRFTISRDNSKNTLYLQMN |
| | | SLRAEDTAVYFCARELGRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 52 | D03 Light | EIVLTQSPATLSLSPGERATLSCRASQSVSSALAWYQQKPGQ |
| | chain | APRLLIYDASKRATGIPARFSGSGSGTDFTLTISSLEPEDFA |
| | | VYYCQQRSKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 53 | B02 Heavy | QVELVESGGGVVQPGRSQRLSCAASGFTFSSTGMHWVRQAPG |
| | chain | KGLEWVAYIWFDGSSTYYADSVRGRFTISRDNSKNTLYLQMN |
| | | SLRAEDTAVYFCARELNRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 54 | B02 Light | EIVLTQSPATLSLSPGERATLSCRASQSVSSYLAWYQQKPGQ |
| | chain | APRLLIYDASKRATGIPARFSGSGSGTDFTLTISSLEPEDFA |
| | | VYYCQQRSKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 55 | B09 Heavy | QVELVESGGGVVQPGRSQRLSCAASGFTFSSHGMHWVRQAPG |
| | chain | KGLEWVAIIAGDASTTYYADSVRGRFTISRDNSKNTLYLOMN |
| | | SLRAEDTAVYFCARELGRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 56 | B09 Light | EIVLTQSPATLSLSPGERATLSCRASQSVSSYLAWYQQKPGQ |
| | chain | APRLLIYDASKRATGIPARFSGSGSGTDFTLTISSLEPEDFA |
| | | VYYCQQRSKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 57 | E01 Heavy | QVELVESGGGVVQPGRSQRLSCAASGFTFSSYGMHWVRQAPG |
| | chain | KGLEWVAIIWFDGSSTYYADSVRGRFTISRDNSKNTLYLQMN |
| | | SLRAEDTAVYFCARELGRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 58 | E01 Light | EIVLTQSPATLSLSPGERATLSCRASQSVSSYLAWYQQKPGQ |
| | chain | APRLLIYDASKRATGIPARFSGSGSGTDFTLTISSLEPEDFA |
| | | VYYCQQRSKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 59 | B10 Heavy | QVELVESGGGVVQPGRSQRLSCAASGFTFSSYGMHWVRQAPG |
| | chain | KGLEWVATIWFDGSSTYYADSVRGRFTISRDNSKNTLYLQMN |
| | | SLRAEDTAVYFCARELGRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 60 | B10 Light | EIVLTQSPATLSLSPGERATLSCRASQSVSSYLAWYQQKPGQ |
| | chain | APRLLIYDASKRATGIPARFSGSGSGTDFTLTISSLEPEDFA |
| | | VYYCQQRSKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 61 | D10 Heavy | QVELVESGGGVVQPGRSQRLSCAASGFTFSSYGMHWVRQAPG |
| | chain | KGLEWVAIINFDGSSTYYADSVRGRFTISRDNSKNTLYLQMN |
| | | SLRAEDTAVYFCARELGRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 62 | D10 Light | EIVLTQSPATLSLSPGERATLSCRASQSVSSYLAWYQQKPGQ |
| | chain | APRLLIYDASKRATGIPARFSGSGSGTDFTLTISSLEPEDFA |
| | | VYYCQQRSKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 63 | C10 Heavy | QVELVESGGGVVQPGRSQRLSCAASGFTFSSAGMHWVRQAPG |
| | chain | KGLEWVAIIWADGSSTYYADSVRGRFTISRDNSKNTLYLQMN |
| | | SLRAEDTAVYFCARELGRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 64 | C10 Light | EIVLTQSPATLSLSPGERATLSCRASQSVSSYLAWYQQKPGQ |
| | chain | APRLLIYDASKRATGIPARFSGSGSGTDFTLTISSLEPEDFA |
| | | VYYCQQRSKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 65 | A11 Heavy | QVELVESGGGVVQPGRSQRLSCAASGFTFSSYGMHWVRQAPG |
| | chain | KGLEWVAYIWFDGSSTYYADSVRGRFTISRDNSKNTLYLQMN |
| | | SLRAEDTAVYFCARELGRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 66 | A11 Light | EIVLTQSPATLSLSPGERATLSCRASQSVSSYLAWYQQKPGQ |
| | chain | APRLLIYDASKRATGIPARFSGSGSGTDFTLTISSLEPEDFA |
| | | VYYCQQRSKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 67 | F02 Heavy | QVELVESGGGVVQPGRSQRLSCAASGFTFSSHGMHWVRQAPG |
| | chain | KGLEWVATIWFDGSSTYYADSVRGRFTISRDNSKNTLYLQMN |
| | | SLRAEDTAVYFCARELGRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 68 | F02 Light | EIVLTQSPATLSLSPGERATLSCRASQSVSSTLAWYQQKPGQ |
| | chain | APRLLIYDASKRATGIPARFSGSGSGTDFTLTISSLEPEDFA |
| | | VYYCQQRSKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 69 | H11 Heavy | QVELVESGGGVVQPGRSQRLSCAASGFTFSSYGMHWVRQAPG |
| | chain | KGLEWVATIWHDGSSTYYADSVRGRFTISRDNSKNTLYLQMN |
| | | SLRAEDTAVYFCARELGRRYFDLWGRGTLVSVSSASTKGPSV |
| | | FPLAPSSKSTSGGTAALGCLVKDYFPEPVTVSWNSGALTSGV |
| | | HTFPAVLQSSGLYSLSSVVTVPSSSLGTQTYICNVNHKPSNT |
| | | KVDKKVEPKSCDKTHTCPPCPAPELLGGPSVFLFPPKPKDTL |
| | | YITREPEVTCVVVDVSHEDPEVKFNWYVDGVEVHNAKTKPRE |
| | | EQYNSTYRVVSVLTVLHQDWLNGKEYKCKVSNKALPAPIEKT |
| | | ISKAKGQPREPQVYTLPPSRDELTKNQVSLTCLVKGFYPSDI |
| | | AVEWESNGQPENNYKTTPPVLDSDGSFFLYSKLTVDKSRWQQ |
| | | GNVFSCSVMHEALHNHYTQKSLSLSPGK |
| 70 | H11 Light | EIVLTQSPATLSLSPGERATLSCRASQSVSSALAWYQQKPGQ |
| | chain | APRLLIYDASKRATGIPARFSGSGSGTDFTLTISSLEPEDFA |
| | | VYYCQQRGKYPPWTFGQGTKVESKRTVAAPSVFIFPPSDEQL |
| | | KSGTASVVCLLNNFYPREAKVQWKVDNALQSGNSQESVTEQD |
| | | SKDSTYSLSSTLTLSKADYEKHKVYACEVTHQGLSSPVTKSF |
| | | NRGEC |
| 71 | Consensus | SX₁GMH, wherein X₁ is H, Y, A, or T |
| | HCDR1 | |
| 72 | Consensus | X₁IX₂X₃DX₄SX₅TYYADSVRG, wherein X₁ is I, |
| | HCDR2 | T, or Y, X₂ is W, N, or A, X₃ is F, H, A, |
| | | or G, X₄ is G or A, X₅ is S or T |
| 73 | Consensus | ELX₁RRYFDL, wherein X₁ is Gor N |
| | HCDR3 | |
| 74 | Consensus | RASQSVSSX₁LA, wherein X₁ is Y, A, or T |
| | LCDR1 | |
| 75 | Consensus | QQRX₁KX₂PPWT, wherein X₁ is S or G, X₂ is |
| | LCDR3 | Y or W |

1. A method of treating thyroid eye disease in a patient in need thereof comprising administering to the patient an insulin-like growth factor-1 receptor (IGF-1R) antagonist antibody, wherein the IGF-1R antagonist antibody comprises an immunoglobulin heavy chain variable region comprising an HCDR1, an HCDR2, and an HCDR3 and an immunoglobulin light chain variable region comprising an LCDR1, an LCDR2, and an LCDR3, wherein:
(a) HCDR1, HCDR2 and HCDR3 comprise the amino acid sequences of SEQ ID NOs: 30, 35, and 41, respectively, and LCDR1, LCDR2, and LCDR3 comprise the amino acid sequences of SEQ ID NOs: 43, 46, and 48, respectively;
(b) HCDR1, HCDR2 and HCDR3 comprise the amino acid sequences of SEQ ID NOs: 33, 38, and 41, respectively, and LCDR1, LCDR2, and LCDR3 comprise the amino acid sequences of SEQ ID NOs: 45, 46, and 48, respectively;
(c) HCDR1, HCDR2 and HCDR3 comprise the amino acid sequences of SEQ ID NOs: 30, 34, and 41, respectively, and LCDR1, LCDR2, and LCDR3 comprise the amino acid sequences of SEQ ID NOs: 45, 46, and 48, respectively;
(d) HCDR1, HCDR2 and HCDR3 comprise the amino acid sequences of SEQ ID NOs: 31, 35, and 42, respectively, and LCDR1, LCDR2, and LCDR3 comprise the amino acid sequences of SEQ ID NOs: 45, 46, and 48, respectively;
(e) HCDR1, HCDR2 and HCDR3 comprise the amino acid sequences of SEQ ID NOs: 33, 40, and 41, respectively, and LCDR1, LCDR2, and LCDR3 comprise the amino acid sequences of SEQ ID NOs: 45, 46, and 48, respectively;
(f) HCDR1, HCDR2 and HCDR3 comprise the amino acid sequences of SEQ ID NOs: 33, 37, and 41, respectively, and LCDR1, LCDR2, and LCDR3 comprise the amino acid sequences of SEQ ID NOs: 45, 46, and 48, respectively;
(g) HCDR1, HCDR2 and HCDR3 comprise the amino acid sequences of SEQ ID NOs: 32, 36, and 41, respectively, and LCDR1, LCDR2, and LCDR3 comprise the amino acid sequences of SEQ ID NOs: 45, 46, and 48, respectively;
(h) HCDR1, HCDR2 and HCDR3 comprise the amino acid sequences of SEQ ID NOs: 33, 35, and 41, respectively, and LCDR1, LCDR2, and LCDR3 comprise the amino acid sequences of SEQ ID NOs: 45, 46, and 48, respectively;
(i) HCDR1, HCDR2 and HCDR3 comprise the amino acid sequences of SEQ ID NOs: 30, 40, and 41, respectively, and LCDR1, LCDR2, and LCDR3 comprise the amino acid sequences of SEQ ID NOs: 44, 46, and 48, respectively; or
(j) HCDR1, HCDR2 and HCDR3 comprise the amino acid sequences of SEQ ID NOs: 33, 39, and 41, respectively, and LCDR1, LCDR2, and LCDR3 comprise the amino acid sequences of SEQ ID NOs: 43, 46, and 47, respectively.

2. The method of claim 1, wherein the immunoglobulin heavy chain variable region comprises an amino acid sequence that is at least 90% identical to an amino acid sequence selected from SEQ ID NOs: 3, 5, 7, 9, 11, 13, 15, 17, 19, and 21.

3. The method of claim 1, wherein the immunoglobulin light chain variable region comprises an amino acid sequence that is at least 90% identical to an amino acid sequence selected from SEQ ID NOs: 4, 6, 8, 10, 12, 14, 16, 18, 20, and 22.

4. The method of claim 1, wherein:
(a) the immunoglobulin heavy chain variable region comprises the amino acid sequence of SEQ ID NO: 17 and the immunoglobulin light chain variable region comprises the amino acid sequence of SEQ ID NO: 18;
(b) the immunoglobulin heavy chain variable region comprises the amino acid sequence of SEQ ID NO: 7 and the immunoglobulin light chain variable region comprises the amino acid sequence of SEQ ID NO: 8;
(c) the immunoglobulin heavy chain variable region comprises the amino acid sequence of SEQ ID NO: 5 and the immunoglobulin light chain variable region comprises the amino acid sequence of SEQ ID NO: 6;
(d) the immunoglobulin heavy chain variable region comprises the amino acid sequence of SEQ ID NO: 3 and the immunoglobulin light chain variable region comprises the amino acid sequence of SEQ ID NO: 4;
(e) the immunoglobulin heavy chain variable region comprises the amino acid sequence of SEQ ID NO: 9 and the immunoglobulin light chain variable region comprises the amino acid sequence of SEQ ID NO: 10;
(f) the immunoglobulin heavy chain variable region comprises the amino acid sequence of SEQ ID NO: 11 and the immunoglobulin light chain variable region comprises the amino acid sequence of SEQ ID NO: 12;
(g) the immunoglobulin heavy chain variable region comprises the amino acid sequence of SEQ ID NO: 13 and the immunoglobulin light chain variable region comprises the amino acid sequence of SEQ ID NO: 14;
(h) the immunoglobulin heavy chain variable region comprises the amino acid sequence of SEQ ID NO: 15 and the immunoglobulin light chain variable region comprises the amino acid sequence of SEQ ID NO: 16;
(i) the immunoglobulin heavy chain variable region comprises the amino acid sequence of SEQ ID NO: 19 and the immunoglobulin light chain variable region comprises the amino acid sequence of SEQ ID NO: 20; or
(j) the immunoglobulin heavy chain variable region comprises the amino acid sequence of SEQ ID NO: 21 and the immunoglobulin light chain variable region comprises the amino acid sequence of SEQ ID NO: 22.

5. The method of claim 1, wherein the IGF-1R antagonist antibody is a monoclonal antibody.

6. The method of claim 5, wherein the monoclonal antibody is a human antibody.

7. The method of claim 5, wherein the IGF-1R antagonist antibody comprises a constant region from a human IgG immunoglobulin.

8. The method of claim 7, wherein the IGF-1R antagonist antibody comprises a constant region from a human IgG1 immunoglobulin.

9. The method of claim 8, wherein the constant region comprises a M252Y, a S254T, and a T256E substitution according to EU numbering.

10. The method of claim 1, wherein the IGF-1R antagonist antibody comprises an immunoglobulin heavy chain comprising an amino acid sequence that is at least 90% identical to an amino acid sequence selected from SEQ ID NOs: 51, 53, 55, 57, 59, 61, 63, 65, 67, and 69, and an immunoglobulin light chain comprising an amino acid sequence that is at least 90% identical to an amino acid sequence selected from SEQ ID NOs: 52, 54, 56, 58, 60, 62, 64, 66, 68, and 70.

11. The method of claim 10, wherein:
(a) the immunoglobulin heavy chain comprises the amino acid sequence of SEQ ID NO: 51 and the immunoglobulin light chain comprises the amino acid sequence of SEQ ID NO: 52;
(b) the immunoglobulin heavy chain comprises the amino acid sequence of SEQ ID NO: 57 and the immunoglobulin light chain comprises the amino acid sequence of SEQ ID NO: 58;
(c) the immunoglobulin heavy chain comprises the amino acid sequence of SEQ ID NO: 55 and the immunoglobulin light chain comprises the amino acid sequence of SEQ ID NO: 56;
(d) the immunoglobulin heavy chain comprises the amino acid sequence of SEQ ID NO: 53 and the immunoglobulin light chain comprises the amino acid sequence of SEQ ID NO: 54;
(e) the immunoglobulin heavy chain comprises the amino acid sequence of SEQ ID NO: 59 and the immunoglobulin light chain comprises the amino acid sequence of SEQ ID NO: 60;
(f) the immunoglobulin heavy chain comprises the amino acid sequence of SEQ ID NO: 61 and the immunoglobulin light chain comprises the amino acid sequence of SEQ ID NO: 62;
(g) the immunoglobulin heavy chain comprises the amino acid sequence of SEQ ID NO: 63 and the immunoglobulin light chain comprises the amino acid sequence of SEQ ID NO: 64;
(h) the immunoglobulin heavy chain comprises the amino acid sequence of SEQ ID NO: 65 and the immunoglobulin light chain comprises the amino acid sequence of SEQ ID NO: 66;
(i) the immunoglobulin heavy chain comprises the amino acid sequence of SEQ ID NO: 67 and the immunoglobulin light chain comprises the amino acid sequence of SEQ ID NO: 68; or
(j) the immunoglobulin heavy chain comprises the amino acid sequence of SEQ ID NO: 69 and the immunoglobulin light chain comprises the amino acid sequence of SEQ ID NO: 70.

12. The method of claim 1, wherein the IGF-1R antagonist antibody is administered to the patient by subcutaneous injection.

13. The method of claim 1, wherein the IGF-1R antagonist antibody is administered to the patient intravenously.

14. The method of claim 1, wherein the patient is diagnosed with active thyroid eye disease.

15. The method of claim 1, wherein the patient is diagnosed with inactive thyroid eye disease.

16. The method of claim 1, wherein the patient is diagnosed with moderate to severe thyroid eye disease.

17. The method of claim 1, wherein the patient has inconstant or constant diplopia prior to administration of the IGF-1R antagonist antibody.

18. The method of claim 1, wherein the patient has an increase in proptosis of 3 mm or more in at least one eye prior to administration of the IGF-1R antagonist antibody.

19. The method of claim 1, wherein proptosis is reduced by at least 2 mm in at least one eye of the patient following administration of the IGF-1R antagonist antibody.

20. The method of claim 19, wherein proptosis is reduced by at least 3 mm in at least one eye of the patient following administration of the IGF-1R antagonist antibody.

21. The method of claim 1, wherein the severity of diplopia in the patient is reduced following administration of the IGF-1R antagonist antibody.

22. The method of claim 1, wherein the clinical activity score (CAS) of the patient is reduced by at least two points following administration of the IGF-1R antagonist antibody.

23. The method of claim 22, wherein the patient has a CAS of 0 or 1 following administration of the IGF-1R antagonist antibody.

24-69. (canceled)
